(19) United States
(12) Patent Application Publication
(10) Pub. No.: US 2026/0199223 A1
(43) Pub. Date: Jul. 16, 2026

(54) USE OF AN AUREOBASIDIUM PULLULANS EXTRACT ALONE OR IN COMBINATION WITH A C-GLYCOSIDE AS A COSMETIC ACTIVE PRINCIPLE

(71) Applicant: L'OREAL, PARIS (FR)

(72) Inventors: Chloé DELAUNAY, CHEVILLY-LARUE (FR); Elodie VALVERDE, CHEVILLY-LARUE (FR); Audrey GUENICHE, AULNAY-SOUS-BOIS (FR); Delphine GUILLON, CHEVILLY-LARUE (FR); Christian TRAN, AULNAY-SOUS-BOIS (FR); Audrey VALOIS, CHEVILLY-LARUE (FR); Franck JUCHAUX, AULNAY-SOUS-BOIS (FR); Suzy LEVOY, CHEVILLY-LARUE (FR)

(21) Appl. No.: 19/137,519

(22) PCT Filed: Dec. 13, 2023
(86) PCT No.: PCT/EP2023/085695

§ 371 Date: Jun. 10, 2025

(30) Foreign Application Priority Data
Dec. 14, 2022 (FR) FR2213330

(51) Int. Cl. A61K 8/9728; A61K 8/60; A61Q 19/08

(52) U.S. Cl. CPC A61K 8/9728; A61K 8/602; A61Q 19/08

(57) ABSTRACT

The present invention relates to the use of a cosmetic active principle comprising at least one extract of the biomass of yeast of the species Aureobasidium pullulans, characterized in that the extract comprises at least 25% sugars, by weight of dry matter of the extract, or in a composition comprising same, as a cosmetic active principle.

### TECHNICAL FIELD

The present invention relates to the field of caring for keratin materials, in particular the anti-ageing care of keratin materials, and is notably directed towards improving and/or reinforcing the barrier function of the skin.

For the purposes of the present invention, the term “keratin materials” notably denotes the skin, the lips and/or the eyelashes, in particular the skin and/or the lips, and preferably the skin of the body, including scalp skin, and/or the face, and more preferentially of the face.

### PRIOR ART

It is known that human skin consists of two tissues: a surface tissue, the epidermis, and a deep tissue, the dermis.

Skin ageing results from the effects of intrinsic and extrinsic factors on the skin. During the ageing process, a detrimental change in the structure and functions of the skin appears. The main clinical signs due to these modifications of the skin metabolism are the appearance of wrinkles and fine lines, the cause of which is a slackening and loss of the elasticity of the tissues.

Moreover, intrinsic ageing, which brings about the changes in the skin, notably causes slowing-down of the renewal of the skin cells, which is reflected essentially by the appearance of detrimental clinical changes, such as a reduction in the subcutaneous adipose tissue and the appearance of small wrinkles or fine lines, and by histopathological changes, such as an increase in the number and thickness of elastic fibres, a loss of vertical fibres from the membrane of the elastic tissue, and the presence of large irregular fibroblasts in the cells of this elastic tissue.

Natural human epidermis is composed mainly of three types of cells, namely keratinocytes, which form the vast majority, melanocytes and Langerhans cells. Each of these types of cells contributes, via its own functions, towards the essential role played in the body by the skin, notably the role of protecting the body against external attacking factors (the climate, ultraviolet rays, smoking, etc.), which is also known as the “barrier function”.

In general, the skin is an organ which inter alia has an essential physical barrier function which prevents the loss of water from the body to the exterior and which prevents external factors from penetrating to the interior.

The stratum corneum, generated by normal differentiation on healthy skin, fulfils a barrier function, which is the most important role of the epidermis. The main cell of the epidermis is the keratinocyte, which is divided into four layers depending on the level of differentiation, and various structural proteins are expressed. The corneodesmosome is expressed on the upper part of the stratum spinosum and the stratum granulosum, which is stored in the lamellar body and secreted outside the cells of the stratum corneum, linking one keratinocyte to another. The lipids contained in the lamellar body of the epidermal keratinocytes at the boundary of the stratum corneum and the stratum granulosum are secreted between the keratinocytes, forming a lipid cell envelope between the keratinocytes, acting as a permeability barrier of the epidermis in the form of a “bricks and mortar” structure of corneocyte and the surrounding intercellular lipid layer.

If the epidermal keratinocytes reach the stratum granulosum, they synthesize keratohyalin granules, which contain profilaggrin and loricrin. The covalent crosslinking of the constituent proteins such as involucrin and loricrin requires transglutaminase (TGase), which is a calcium-dependent enzyme that catalyses the formation of an intermolecular isopeptide bond between proteins. Of the nine TGases identified in humans, TGases 1, 3 and 5 are known to participate in the formation of the cornified envelopes. TGases 1 and 3 are both activated by limited proteolysis during keratinocyte differentiation. These enzymes contribute in a cooperative manner to the sequential crosslinking of substrates.

If the differentiation of the epidermal keratinocytes is abnormal, it will lead to defects in the skin's barrier function and to skin dehydration, and compromised skin.

Tight junctions are of particular interest because they are located at the centre of this complex barrier system in the outermost viable layer—the stratum granulosum of the interfollicular epidermis and the companion cell layer of the hair follicle- and because they can react very quickly to stimuli.

The tight junction is a crucial organelle for the development and function of most vertebrate organ systems, since it allows epithelia and endothelia to create fluid compartments that are different in composition.

The tight junction forms a continuous permeability barrier between adjacent cells that regulates the flow of molecules through the paracellular space.

It forms the closest contact known in nature between adjacent cells, so close, in fact, that it was once thought to be a fusion between the outer leaflets of adjacent bilayers. It is now known that it is not an absolute seal, but rather that it contains discrete ion-selective pores through the extracellular portion of the junction.

The extracellular portion of the tight junction must carry out two main tasks: to form extremely tight intercellular adhesions that close off a region of extracellular space, and to create regulatable pores for selective molecular screening.

In the prior art, the yeast Aureobasidium pullulans is essentially used for fermenting organic animal or plant matter or for producing a particular polysaccharide, pullulan.

Cosmetic compositions comprising an A. pullulans culture supernatant or plant extracts fermented by A. pullulans are also known, but no teaching describes the use of an A. pullulans extract containing at least 25% sugars according to the present invention, in particular for an anti-ageing effect and/or an effect of improving and/or reinforcing the skin's barrier function.

The Applicant has discovered, very surprisingly, that the use of biomass, and more particularly an extract of the biomass of yeast of the species Aureobasidium pullulans, notably as identified in the microbiota of rose bushes grown on the Valensole plateau in France, is of major interest as a cosmetic active principle, more particularly in the field of keratin material care, notably for combating the signs of skin ageing and for improving and/or reinforcing the skin's barrier function.

The Applicant has thus discovered, very surprisingly, that a combination comprising at least one extract of the biomass of yeast of the species Aureobasidium pullulans and at least one C-glycoside, makes it possible to effectively combat the signs of skin ageing and to improve and/or reinforce the skin's barrier function.

It has thus been demonstrated, as illustrated in the following examples, that this extract of the biomass of yeast of the species Aureobasidium pullulans is effective both on its own and in combination with C-β-D-xylopyranoside-2-hydroxypropane:
on the ageing of cells in the epidermal compartment,
on the migration of aged keratinocytes,
on the tight junctions, and
on keratinocyte differentiation,
   which has hitherto never been described.

These efficiencies support a strengthening of the skin barrier. This reinforced skin barrier contributes to the quality of the skin's surface for radiance, uniformity of the complexion and smooth and soft skin.

This extract of the biomass of yeast of the species Aureobasidium pullulans and this combination also find their application in compositions, preferably cosmetic compositions, intended for preventing and/or treating the signs of skin ageing; preferably for preventing and/or treating the cutaneous signs associated with skin displaying impairments in its viscoelastic or biomechanical properties, skin displaying impairments in the cohesion of its tissues, thinned skin, wrinkled skin and/or skin displaying impairments in its surface appearance. Said extract and said combination are preferably used topically.

This extract of the biomass of yeast of the species Aureobasidium pullulans and also this combination may more particularly allow the skin's extensibility, tonicity, firmness, suppleness, density and/or elasticity properties to be maintained and/or restored.

Finally, the formulation of environmentally friendly cosmetic products has become a major challenge for meeting new consumer expectations, in particular regarding natural and/or eco-friendly products, i.e. products whose design and development take into account their environmental impacts.

It is thus common practice to seek to replace synthetic or environmentally unfriendly compounds present in cosmetic compositions with natural ingredients and/or ingredients of natural origin.

### DISCLOSURE OF THE INVENTION

There is thus still a need for cosmetic active principles which are natural and/or of natural origin, which may be used in cosmetic compositions, alone or in combination with other cosmetic active principles, in particular for combating skin ageing, in particular for improving and/or reinforcing the skin's barrier function.

The need also remains to have compositions that are compatible with the current consumer demands, notably regarding the environment.

The present invention is specifically directed towards meeting all or some of these needs.

### SUMMARY OF THE INVENTION

Thus, according to one of its aspects, the invention proposes the use of a cosmetic active principle comprising at least one extract of the biomass of yeast of the species Aureobasidium pullulans, characterized in that the extract comprises at least 25% sugars, by weight of dry matter of the extract, or in a composition comprising same, as a cosmetic active principle.

According to another of its aspects, the invention proposes a composition, notably a non-therapeutic cosmetic composition, comprising at least one cosmetic active principle comprising at least one extract of the biomass of yeast of the species Aureobasidium pullulans, notably as defined below, and at least one C-glycoside, notably as defined below, in particular present in a mass ratio as defined below.

A composition according to the invention is in particular used for preventing and/or treating the signs of skin ageing and/or for reinforcing and/or improving the skin's barrier function. Finally, according to another of its aspects, the invention proposes a non-therapeutic cosmetic process for keratin materials, comprising at least one step of applying onto said keratin materials, preferably onto the skin, a composition as defined above and detailed hereinbelow.

The cosmetic processes for caring for keratin materials, in particular the skin, are non-therapeutic.

Thus, the present application also relates to the use of a composition according to the invention for combating the signs of skin ageing and/or for reinforcing and/or improving the skin's barrier function.

The term “biomechanical properties of the skin” means herein the stretchability, tonicity, firmness, suppleness and/or elasticity properties of the skin.

The term “signs of skin ageing” means herein any modification of the outer appearance of the skin due to ageing, whether it is chronobiological and/or extrinsic ageing, in particular photoinduced or hormonal ageing; among these signs, it is possible to distinguish:
wrinkled skin, which is notably reflected by the appearance of wrinkles and/or fine lines;
skin displaying impairment of its viscoelastic or biomechanical properties, or skin exhibiting a lack of elasticity and/or of stretchability and/or of firmness and/or of suppleness and/or of tonicity, which is reflected in particular by wizened, flaccid, slack or saggy skin;
skin displaying impairment of the cohesion of its tissues;
thinned skin; and
skin displaying impairment of its surface appearance, which is notably reflected by impairment of the grain of the skin, for example roughness.

For the purposes of the invention, “Aureobasidium pullulans” means any yeast of the Dothioraceae family, of the genus Aureobasidium and of the species Aureobasidium pullulans.

Other characteristics, variants and advantages of the compositions in accordance with the invention will emerge more clearly on reading the description and the examples that follow.

### DETAILED DESCRIPTION

Extract of Aureobasidium pullulans

The yeast Aureobasidium pullulans, also known as black yeast, is ubiquitous, multi-extremotolerant and occurs either as a filamentous fungus or in unicellular form. A. pullulans is well known as a natural epiphyte of many plant species, such as apples and grapes. For several years, it has been used in biotechnology for the production of several enzymes or for combating certain plant diseases, such as apple blight.

Thus, the cosmetic active principle in accordance with the present invention comprises at least one extract derived from the biomass of yeast of the species Aureobasidium pullulans, comprising at least 25% sugars, by weight of dry matter of the extract.

Advantageously, the Aureobasidium pullulans yeast may be collected from roses, preferably grown on the Valensole plateau (France); more preferentially, the yeast is collected from the flowers and/or thorns and/or roots of Rosa sp.

Preferably, Aureobasidium pullulans is isolated from rose bushes (Rosa sp.).

The Rosa genus includes more than 200 species, among which mention may be made of Rosa alba, Rosa alpina, Rosa canina, Rosa cinnamonea, Rosa gallica, Rosa repens, Rosa rubrifolia, Rosa rubiginosa, Rosa sempervirens, Rosa spinosissima, Rosa stylosa, Rosa tomentosa, or even Rosa villosa, Rosa floribunda.

More preferentially, Aureobasidium pullulans is isolated from a hybrid rose, such as a rose of the species Rosa floribunda, better still a hybrid rose of the variety Rosa floribunda delflobla notably available commercially under the trade name Rose Lancôme® from the company Delbard (France). The Rosa floribunda delflobla variety is also described in US 2020/0178431.

In a preferred embodiment, the Aureobasidium pullulans yeast in accordance with the present invention is not genetically modified.

In a preferred embodiment, the Aureobasidium pullulans yeast in accordance with the present invention is derived from a strain registered in a yeast collection (CIRM), under CLIB number 2138, on 13 Jan. 2021.

For the purposes of the invention, the term “Aureobasidium pullulans extract” means an extract comprising at least one set of molecules, preferably an extract of the biomass of the yeast Aureobasidium pullulans comprising at least sugars representing at least 25% by weight of dry matter relative to the total weight of the extract, obtained via any extraction process well known to those skilled in the art, for example by induced autolysis, sonication, homogenization, chemical hydrolysis or enzymatic hydrolysis. Such extraction processes are described in the publications Varelas V. et al., Drug Test Anal. 2016 January and Du L. et al. Molecules. 2020 Jan. 23.

The extract in accordance with the invention is not an extract of the culture supernatant of Aureobasidium pullulans.

For the purposes of the invention, the term “yeast culture supernatant extract” means an extract that is not an extract of the Aureobasidium pullulans culture biomass.

According to one embodiment, the yeast Aureobasidium pullulans is ubiquitous, and so may be present in several plant species. Preferentially, the Aureobasidium pullulans yeast is isolated from rose bushes, more preferentially from the flowers and/or thorns and/or roots of Rosa sp.

The Aureobasidium pullulans biomass is notably produced from the yeast previously obtained. This step is performed according to the yeast culture method in a medium that is suitable for their growth, for example a suitable culture medium, in a manner that is conventional for those skilled in the art. An example of culture medium suitable cultivation of the Aureobasidium pullulans biomass is recited in “β-Glucan derived from Aureobasidium pullulans is effective for the prevention of influenza in mice”, Muramatsu D, Iwai A, Aoki S, Uchiyama H, Kawata K, Nakayama Y, Nikawa Y, Kusano K, Okabe M, Miyazaki T, PLOS One. 2012. Preferably, a suitable culture comprises at least one carbon source such as carbohydrates, and at least one nitrogen source. Preferably cultivation of the Aureobasidium pullulans biomass is performed between 20° C. to 30° C. Once the biomass has been obtained, an extraction step is performed to obtain the active molecules, preferentially an extraction of the sugars.

The extract in accordance with the invention comprises at least 25% sugars by weight of the dry matter of the extract. The extract in accordance with the invention may also comprise peptides and mineral ash.

Thus, the extract in accordance with the invention comprises sugars, which represent at least 25% of the extract, by weight of the dry matter of the extract. Preferentially, the extract in accordance with the invention comprises at least 45% sugars, by weight of the dry matter of the extract.

The total sugar content in said cosmetic active principle may be determined by the Dubois method (Dubois M. et al., Analytical Chemistry, 28, 3, 350-356, 1956). The total sugar content in the cosmetic active principle in accordance with the invention is expressed as a percentage relative to the dry matter.

The size of the sugars present in the extract may be characterized by HPLC/RI chromatography. They may be in the form of monosaccharides or in the form of oligosaccharides; preferentially, the oligosaccharides have a molar mass of less than 1800 Da.

Particularly preferably, oligosaccharides predominate in the extract according to the present invention. Finally, according to a particularly preferred embodiment, the sugars of the extract are composed of at least 80% oligosaccharides, by weight of dry matter of the sugars.

Preferentially, the oligosaccharides in the extract are alpha-linked glucose oligosaccharides and/or beta-linked glucose oligosaccharides.

The alpha-linked glucose oligosaccharides are oligomers consisting of D-glucose linked by alpha linkages. These oligosaccharides present in the cosmetic active principle in accordance with the invention may have a degree of polymerization (DP) of between 2 and 10 units. Moreover, these oligosaccharides may have a molar mass of less than 1800 Daltons. Preferentially, they represent between 1.5 and 4 g/L.

Beta-linked glucose oligosaccharides are oligosaccharides consisting of D-glucose linked by beta linkages. These oligosaccharides present in the cosmetic active principle in accordance with the invention have a degree of polymerization of between 2 and 10 units, and have a molar mass of less than 1800 Daltons. Preferentially, they represent between 1.5 and 4 g/L. Thus, the cosmetic active principle in accordance with the invention comprises sugars of oligosaccharide structures having a degree of polymerization (DP) of less than 10, i.e. a molar mass of less than 1800 Daltons.

The extract in accordance with the invention also comprises peptides. Preferentially, these represent at least 30% by weight of the dry matter of the extract. The peptide content may be determined by measuring the total nitrogen content according to the Kjeldhal method (reference: Official method of analysis of the A.O.C., 12th ed. W Horwitz, E. D., New York, 15-60, 1975). The peptides have a molar mass of less than 2000 Da. The distribution and molar mass of peptides may be determined by FPLC steric exclusion chromatography (Fast Protein Liquid Chromatography), the amount of each peptide fraction is determined by spectrophotometric assay using the Lowry method (Lowry et al., Protein measurement with the Folin reagent, J. Biol. Chem., 193, 265-275, 1951).

When the extract in accordance with the invention comprises mineral ash, the ash content is preferentially between 8% and 18% by weight relative to the dry matter of the extract, even more preferentially between 10% and 15%. The crude ash content may be determined by weighing the residues from incineration of the samples at 550° C. in a muffle furnace. The nature of the minerals contained in the ash may be determined by optical emission spectrometry (ICP/OES). The chloride ions contained in the ash are determined by titration with silver nitrate. Preferentially, the mineral ash contained in the extract in accordance with the invention comprises calcium, phosphorus, chloride ions, magnesium, sulfur and sodium. The dry matter content of the extract may be determined by weighing the residues from drying the extract in accordance with the invention at 105° C. in an oven until a constant weight is obtained. Preferentially, the extract in accordance with the invention in liquid form has a dry matter content of: 10 g/L to 40 g/L, even more preferentially from 17 g/L to 26 g/L.

The cosmetic active principle in accordance with the invention may be in liquid form or in solid form.

When it is in liquid form, the cosmetic active principle in accordance with the invention is preferentially the extract as described previously as such. It is in the form of a clear, very pale yellow liquid with a faint odour. It may, however, be coloured and/or decolourized via any process known to those skilled in the art. The cosmetic active principle may also be combined with stabilizers or preserving agents.

When it is in solid form, the cosmetic active principle in accordance with the invention preferentially consists of the Aureobasidium pullulans extract as described previously and of a support chosen from maltodextrin, gum arabic, soya lecithin or isomalt. According to a particularly suitable embodiment, the extract represents at least 10% by weight of the cosmetic active principle and the support not more than 90% by weight of the cosmetic active principle.

In the case of a solid form in which the cosmetic active principle is combined with a support, the protein, sugar and ash contents in the cosmetic active principle are modified, the support generally consisting predominantly of sugars.

The cosmetic active principle according to the invention may optionally be incorporated, for the use according to the present invention, into a cosmetic composition, notably a composition comprising at least 0.0005% by weight of dry matter of said cosmetic active principle. In particular, the composition is in a form that is suitable for topical application, such as a cream or a lotion.

### Extraction Process

The extract constituting or contained in the cosmetic active principle in accordance with the invention may be obtained via any process comprising at least one step of extracting the biomass of Aureobasidium pullulans yeast. Extraction of the yeast biomass does not consist in recovering the supernatant of the yeast culture.

Prior to the process for obtaining the extract as such, the Aureobasidium pullulans biomass has to be produced. This step is performed according to the yeast culture method in a medium that is suitable for their growth, for example a suitable culture medium, in a manner that is conventional for those skilled in the art. Once the biomass has been obtained, an extraction step may be performed to obtain the active molecules, preferentially sugars. The extraction step may be performed by any means known to those skilled in the art, for example by induced autolysis, sonication, homogenization, enzymatic lysis, chemical hydrolysis or enzymatic hydrolysis.

For example, the cosmetic active principle in accordance with the invention may be obtained by performing the following steps:
a. cultivation of the Aureobasidium pullulans biomass in a culture medium, preferentially a suitable culture medium,
b. dissolution of at least 50 g/L of the Aureobasidium pullulans biomass in water,
c. extraction, preferentially extraction of the sugars,
d. heat treatment, preferably between 80° C. and 90° C.,
e. separation of the soluble and insoluble phases, and recovery of the soluble phase,
f. purification by molecular sorting, and optional decolourization and deodorization, and
g. optional concentration and sterilizing filtration.

Step d is a conventional heat treatment step from those skilled persons in the art used for stabilization of the extract obtained further to step c. In particular, this treatment avoids precipitation or unwanted natural enzymatic activity from certain molecules such as proteins possibly present in the said extract.

Separation of the soluble and insoluble phases is performed by any means known to a person skilled in the art, for example by centrifugation, filtration or decantation. Preferentially, the separation of the soluble and insoluble phases is performed to recover the soluble phase containing inter alia soluble sugars, such as oligosaccharides.

Optionally, the process comprises a filtration step after recovery of the soluble phase to remove any particles still in suspension. This filtration step thus allows the recovered soluble phase to be purified and is performed so as to remove the high molecular weight molecules. The product obtained at this stage may optionally be further concentrated and/or purified, preferentially by successive ultrafiltration steps through filters of different porosity, keeping the filtrates at each stage, and/or by a chromatographic type method.

The product obtained after filtration, before or after concentration and sterilizing filtration, is an extract of Aureobasidium pullulans, and consists of a first form of the cosmetic active principle in accordance with the invention, which is then in liquid form.

The extract obtained may then be dried and optionally combined with a support, to be in solid form. This phase may be performed by carrying out the following steps:
an atomization support, preferably maltodextrin, is added to the extract of Aureobasidium pullulans, to a maximum of 90% (by mass/volume);
this solution is then concentrated under vacuum;
any bacteria that may be present are removed by heat treatment;
atomization allows a powder to be obtained.

The steps of the processes described above, taken individually, are common in the field of extracting active principles from natural raw materials and a person skilled in the art is capable of adjusting the reaction parameters thereof on the basis of his general knowledge.

### Use and Cosmetic Process According to the Invention

According to one embodiment of the invention, the use according to the invention is characterized in that it is intended for preventing and/or treating the signs of skin ageing and/or for reinforcing and/or improving the skin's barrier function.

According to another embodiment of the invention, the use according to the invention is characterized in that it is intended for moisturizing the skin and/or for improving the quality of the skin surface, in particular for improving the radiance of the skin and/or for improving the uniformity of the complexion and/or for reducing the microrelief of the skin.

According to yet another embodiment, the use is characterized in that said cosmetic active principle is in combination with at least one C-glycoside, notably as defined hereinbelow.

As mentioned previously, the present invention relates to a cosmetic process for keratin materials, comprising at least one step of applying onto said keratin materials, preferably onto the skin, a composition according to the invention, as detailed hereinbelow.

According to a particular embodiment, the cosmetic process is characterized in that it is intended for preventing and/or treating the signs of skin ageing and/or for reinforcing and/or improving the skin's barrier function.

According to a particular embodiment, the cosmetic process is characterized in that it is intended for moisturizing the skin and/or improving the quality of the skin surface, in particular improving the radiance of the skin and/or improving the uniformity of the complexion and/or reducing the microrelief of the skin.

The cosmetic process and use according to the invention are performed by topical administration of a composition comprising a cosmetic active principle comprising at least one extract conforming to the invention, optionally in combination with a C-glycoside.

Topical administration consists of the external application to the skin of such a cosmetic composition according to the usual techniques for the use of these compositions. By way of illustration, the cosmetic process or use according to the invention may be performed by topical, for example daily, application of such a composition, which may for example be formulated in the form of a cream, a gel, a serum, a lotion, an emulsion, a cleansing milk, a stick or an aftersun composition, preferably in the form of an emulsion. According to one embodiment, the application is repeated, for example, once or twice a day for one or more days and generally over an extended period of at least 4 weeks, or even 4 to 15 weeks, with, where appropriate, one or more periods of stoppage.

According to another embodiment, the application is daily (once a day) and generally over an extended period of at least 4 weeks, or even 4 to 15 weeks, with, where appropriate, one or more periods of stoppage.

Specifically, as emerges from the examples hereinbelow, the extract in accordance with the present invention has been shown to obtain an improved effect on the pathways of ageing metabolism (Example 4) or an improved effect in stimulating the migration of aged keratinocytes and thus anti-ageing efficacy (Example 5).

Furthermore, as is also apparent from the examples given hereinbelow, the extract in accordance with the present invention was tested, alone or in combination with a C-glycoside, notably on epidermal markers, notably filaggrin, loricrin, claudin-1, ZO-1, CK10, TGK (encoded by the gene TGM1) and involucrin (Examples 6 and 8).

In particular, transglutaminase K (TGK), filaggrin and involucrin, CK10 and loricrin are markers of the differentiation of stratum corneum keratinocytes.

Filaggrin and TGK are moreover markers of the skin's barrier function.

Claudin-1, occludin and ZO-1 (zonula occludens-1) are markers of tight junctions.

A composition comprising the extract in accordance with the present invention, alone or in combination with a C-glycoside, as described previously, was moreover clinically tested and demonstrated beneficial effects on the skin, in particular on the barrier function and the microrelief (Example 7).

The extract in accordance with the present invention, alone or in combination with a C-glycoside, thus makes it possible to stimulate in the skin the production of tight junction proteins, inducing improved intercellular cohesion, and stimulates in the skin the production of proteins involved in keratinocyte differentiation.

Cosmetic Composition Comprising the Combination of an Extract of Aureobasidium pullulans and a C-Glycoside

### C-Glycoside

The C-glycoside (or C-glycoside derivative) in accordance with the invention is preferably of the general formula (I) below:

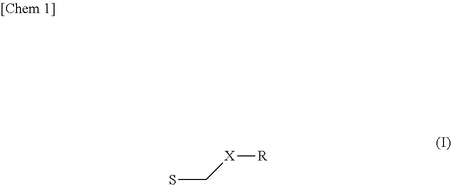

in which:
   R represents:
   a saturated linear C1 to C20, preferably C1 to C10, or unsaturated C2 to C20, preferably C3 to C10, alkyl radical, or a saturated or unsaturated, branched or cyclic C3 to C20, preferably C4 to C10, alkyl radical;
   a saturated linear C1 to C20, preferably C2 to C10, or unsaturated C2 to C20, preferably C2 to C10, or branched or cyclic, saturated or unsaturated, C3 to C20, preferably C4 to C10, hydrofluoro- or perfluoroalkyl radical;
   a phenyl or benzyl radical,
   the hydrocarbon-based chain constituting said radicals possibly being, where appropriate, interrupted with 1, 2, 3 or more heteroatoms chosen from: an oxygen, a sulfur, a nitrogen, a silicon, a halogen atom,
   and possibly being optionally substituted with at least one radical chosen from: —OR4, —SR4, —NR4R5, —COOR4, —CONHR4, —CN, a C1 to C6 hydrofluoro- or perfluoroalkyl radical, and/or a C3 to C8 cycloalkyl radical, and/or at least one, optionally substituted, C5 to C18 cycloalkyl, aryl or heterocyclic radical,
   where R4 and R5 may represent, independently of each other, a hydrogen atom, or a linear, saturated C1 to C30, preferably C3 to C12, or unsaturated C2 to C30, preferably C3 to C12, or branched or cyclic, saturated or unsaturated, C3 to C30, preferably C4 to C12, alkyl, perfluoroalkyl or hydrofluoroalkyl radical; or a C6 to C10 aryl radical,
X represents a radical chosen from —CO—, —CH(OH)— and —CH(NH₂)— and preferentially a —CH(OH)— group;
S represents a monosaccharide or a polysaccharide including up to 20 sugar units and preferably up to 6 sugar units, in pyranose and/or furanose form and of L and/or D series, said mono- or polysaccharide possibly being substituted with a mandatorily free hydroxyl group, and optionally one or more optionally protected amine functions, and
the S—CH₂—X bond represents a bond of C-anomeric nature, which may be α or β, and also the physiologically acceptable salts thereof, the solvates thereof, such as hydrates, and the isomers thereof.

In the context of the present invention, the term “halogen” means chlorine, fluorine, bromine or iodine.

The term “aryl” denotes an aromatic ring such as phenyl, optionally substituted with one or more C1-C4 alkyl radicals.

The term “C3 to C8 cycloalkyl” denotes an aliphatic ring containing from 3 to 8 carbon atoms, including for example cyclopropyl, cyclopentyl and cyclohexyl.

Among the alkyl groups that are suitable for use in the invention, mention may notably be made of methyl, ethyl, isopropyl, n-propyl, n-butyl, t-butyl, isobutyl, sec-butyl, pentyl, n-hexyl, cyclopropyl, cyclopentyl, cyclohexyl and allyl.

A C-glycoside may correspond to formula (I) for which S may represent a monosaccharide or a polysaccharide containing up to 6 sugar units, in pyranose and/or furanose form and of L and/or D series, said mono- or polysaccharide having at least one mandatorily free hydroxyl function and/or optionally one or more mandatorily protected amine functions, X and R otherwise retaining all the definitions given previously.

Advantageously, a monosaccharide in accordance with the invention may be chosen from D-glucose, D-galactose, D-mannose, D-xylose, D-lyxose, L-fucose, L-arabinose, L-rhamnose, D-glucuronic acid, D-galacturonic acid, D-iduronic acid, N-acetyl-D-glucosamine, N-acetyl-D-galactosamine and advantageously denotes D-glucose, D-xylose, N-acetyl-D-glucosamine or L-fucose, and more preferentially D-xylose.

Advantageously, a polysaccharide in accordance with the invention containing up to 6 sugar units may be chosen from D-maltose, D-lactose, D-cellobiose, D-maltotriose, a disaccharide combining a uronic acid chosen from D-iduronic acid or D-glucuronic acid with a hexosamine chosen from D-galactosamine, D-glucosamine, N-acetyl-D-galactosamine, N-acetyl-D-glucosamine, an oligosaccharide containing at least one xylose which may advantageously be chosen from xylobiose, methyl-β-xylobioside, xylotriose, xylotetraose, xylopentaose and xylohexaose and notably xylobiose which is composed of two xylose molecules linked by a 1-4 linkage.

More preferentially, S may represent a monosaccharide chosen from D-glucose, D-xylose, L-fucose, D-galactose and D-maltose, and better still D-xylose.

Advantageously, a C-glycoside corresponding to formula (I) may be used in which R represents a linear, saturated C1 to C20, preferably C1 to C10, or unsaturated C2 to C20, preferably C3 to C10, alkyl radical, or a branched or cyclic, saturated or unsaturated C3 to C20, preferably C4 to C10, alkyl radical, and optionally substituted as described previously, S and X otherwise retaining all the definitions given previously.

More preferentially, R may denote a linear C1-C4 and preferably C1-C2 radical, optionally substituted with —OH, —COOH or —COOR″2, R″2 being a saturated C1-C4 alkyl radical, notably methyl. Better still, R may denote an unsubstituted linear C1-C4 and preferably C1-C2 alkyl radical, such as methyl.

According to a preferred embodiment, among the C-glycosides of formula (I), the ones that are used are those for which:
R represents a linear, saturated C1 to C20, preferably C1 to C10, or unsaturated C2 to C20, preferably C3 to C10, alkyl radical or a branched or cyclic, saturated or unsaturated, C3 to C20, preferably C4 to C10, alkyl radical optionally substituted as described previously;
S represents a monosaccharide as described previously, preferably D-glucose, D-xylose, N-acetyl-D-glucosamine or L-fucose, more preferentially D-xylose;
X represents a radical chosen from —CO—, —CH(OH)— and —CH(NH2)-, and preferably a —CH(OH)— group.

According to a preferred embodiment, use is made of a C-glycoside of formula (I) for which:
R denotes a linear C1-C4 and preferably C1-C3 radical, optionally substituted with —OH, —COOH or —COOR″2, R″2 being a saturated C1-C4 alkyl radical, such as methyl;
S represents a monosaccharide as described previously, preferably D-glucose, D-xylose, N-acetyl-D-glucosamine or L-fucose, more preferentially D-xylose;
X represents a radical chosen from —CO—, —CH(OH)— and —CH(NH2)-, and preferably a —CH(OH)— group.

According to a preferred embodiment, use may be made of a C-glycoside of formula (I) for which:
R denotes an unsubstituted linear C1-C4 and preferably C1-C2 alkyl radical, such as methyl;
S represents a monosaccharide as described previously, preferably D-glucose, D-xylose, N-acetyl-D-glucosamine or L-fucose, more preferentially D-xylose;
X represents a group chosen from —CO—, —CH(OH)— and —CH(CH2)- and —CH(OH)—, preferentially a —CH(OH)— group;

The acceptable salts for the non-therapeutic use of the compounds described in the present invention include conventional non-toxic salts of said compounds, such as those formed from organic or inorganic acids. By way of example, mention may be made of the salts of mineral acids, such as sulfuric acid, hydrochloric acid, hydrobromic acid, hydriodic acid, phosphoric acid or boric acid. Mention may also be made of the salts of organic acids, which may include one or more carboxylic, sulfonic or phosphonic acid groups. They may be linear, branched or cyclic aliphatic acids, or alternatively aromatic acids. These acids may also include one or more heteroatoms chosen from O and N, for example in the form of hydroxyl groups. Mention may be made especially of propionic acid, acetic acid, terephthalic acid, citric acid and tartaric acid.

When the compound of formula (I) comprises an acidic group, neutralization of the acid group(s) may be performed with a mineral base, such as LiOH, NaOH, KOH, Ca(OH)2, NH4OH, Mg(OH)2 or Zn(OH)2; or with an organic base such as a primary, secondary or tertiary alkylamine, for example triethylamine or butylamine. This primary, secondary or tertiary alkylamine may comprise one or more nitrogen and/or oxygen atoms and may thus comprise, for example, one or more alcohol functions; mention may be made especially of 2-amino-2-methylpropanol, triethanolamine, 2-dimethylaminopropanol and 2-amino-2-(hydroxymethyl)-1,3-propanediol. Mention may also be made of lysine or 3-(dimethylamino) propylamine.

The solvates that are acceptable for the compounds described in the present invention comprise conventional solvates such as those formed during the final step of preparation of said compounds due to the presence of solvents. Examples that may be mentioned include solvates due to the presence of water or of linear or branched alcohols, such as ethanol or isopropanol.

Preferably, the C-glycosides of formula (I) used in accordance with the invention are chosen from:
1. C-β-D-xylopyranoside-n-propan-2-one;
2. C-α-D-xylopyranoside-n-propan-2-one;
3. 1-[2-(3-hydroxypropylamino) propyl]-C-β-D-xylopyranose;
4. 1-[2-(3-hydroxypropylamino) propyl]-C-α-D-xylopyranose;
5. C-β-D-xylopyranoside-2-hydroxypropane;
6. C-α-D-xylopyranoside-2-hydroxypropane;
7. C-β-D-xylopyranoside-2-aminopropane;
8. C-α-D-xylopyranoside-2-aminopropane;
9. C-β-D-xylopyranoside-2-phenylaminopropane;
10. C-α-D-xylopyranoside-2-phenylaminopropane;
11. ethyl ester of 3-methyl-4-(C-β-D-xylopyranoside)-butyric acid;
12. ethyl ester of 3-methyl-4-(C-α-D-xylopyranoside)-butyric acid;
13. 6-(C-β-D-xylopyranoside)-5-ketohexanoic acid;
14. 6-(C-α-D-xylopyranoside)-5-ketohexanoic acid;
15. 6-(C-β-D-xylopyranoside)-5-hydroxyhexanoic acid;
16. 6-(C-α-D-xylopyranoside)-5-hydroxyhexanoic acid;
17. 6-(C-β-D-xylopyranoside)-5-aminohexanoic acid;
18. 6-(C-α-D-xylopyranoside)-5-aminohexanoic acid;
19. 6-(C-β-D-xylopyranoside)-5-phenylaminohexanoic acid;
20. 6-(C-α-D-xylopyranoside)-5-phenylaminohexanoic acid;
21. 1-(C-β-D-xylopyranoside) hexane-2,6-diol;
22. 1-(C-α-D-xylopyranoside) hexane-2,6-diol;
23. 5-(C-β-D-xylopyranoside)-4-ketopentanoic acid;
24. 5-(C-α-D-xylopyranoside)-4-ketopentanoic acid;
25. 5-(C-β-D-xylopyranoside)-4-hydroxypentanoic acid;
26. 5-(C-α-D-xylopyranoside)-4-hydroxypentanoic acid;
27. 5-(C-β-D-xylopyranoside)-4-aminopentanoic acid;
28. 5-(C-α-D-xylopyranoside)-4-aminopentanoic acid;
29. 5-(C-β-D-xylopyranoside)-4-phenylaminopentanoic acid;
30. 5-(C-α-D-xylopyranoside)-4-phenylaminopentanoic acid;
31. 1-(C-β-D-xylopyranoside) pentane-2,5-diol;
32. 1-(C-α-D-xylopyranoside) pentane-2,5-diol;
33. 1-(C-β-D-fucopyranoside) propan-2-one;
34. 1-(C-α-D-fucopyranoside) propan-2-one;
35. 1-(C-β-L-fucopyranoside) propan-2-one;
36. 1-(C-α-L-fucopyranoside) propan-2-one;
37. 1-(C-β-D-fucopyranoside)-2-hydroxypropane;
38. 1-(C-α-D-fucopyranoside)-2-hydroxypropane;
39. 1-(C-β-L-fucopyranoside)-2-hydroxypropane;
40. 1-(C-α-L-fucopyranoside)-2-hydroxypropane;
41. 1-(C-β-D-fucopyranoside)-2-aminopropane;
42. 1-(C-α-D-fucopyranoside)-2-aminopropane;
43. 1-(C-β-L-fucopyranoside)-2-aminopropane;
44. 1-(C-α-L-fucopyranoside)-2-aminopropane;
45. 1-(C-β-D-fucopyranoside)-2-phenylaminopropane;
46. 1-(C-α-D-fucopyranoside)-2-phenylaminopropane;
47. 1-(C-β-L-fucopyranoside)-2-phenylaminopropane;
48. 1-(C-α-L-fucopyranoside)-2-phenylaminopropane;
49. ethyl ester of 3-methyl-4-(C-β-D-fucopyranoside)-butyric acid;
50. ethyl ester of 3-methyl-4-(C-α-D-fucopyranoside)-butyric acid;
51. ethyl ester of 3-methyl-4-(C-β-L-fucopyranoside)-butyric acid;
52. ethyl ester of 3-methyl-4-(C-α-L-fucopyranoside)-butyric acid;
53. 6-(C-β-D-fucopyranoside)-5-ketohexanoic acid;
54. 6-(C-α-D-fucopyranoside)-5-ketohexanoic acid;
55. 6-(C-β-L-fucopyranoside)-5-ketohexanoic acid;
56. 6-(C-α-L-fucopyranoside)-5-ketohexanoic acid;
57. 6-(C-β-D-fucopyranoside)-5-hydroxyhexanoic acid;
58. 6-(C-α-D-fucopyranoside)-5-hydroxyhexanoic acid;
59. 6-(C-β-L-fucopyranoside)-5-hydroxyhexanoic acid;
60. 6-(C-α-L-fucopyranoside)-5-hydroxyhexanoic acid;
61. 6-(C-β-D-fucopyranoside)-5-aminohexanoic acid;
62. 6-(C-α-D-fucopyranoside)-5-aminohexanoic acid;
63. 6-(C-β-L-fucopyranoside)-5-aminohexanoic acid;
64. 6-(C-α-L-fucopyranoside)-5-aminohexanoic acid;
65. 1-(C-β-D-fucopyranoside) hexane-2,6-diol;
66. 1-(C-α-D-fucopyranoside) hexane-2,6-diol;
67. 1-(C-β-L-fucopyranoside) hexane-2,6-diol;
68. 1-(C-α-L-fucopyranoside) hexane-2,6-diol;
69. 5-(C-β-D-fucopyranoside)-4-ketopentanoic acid;
70. 5-(C-α-D-fucopyranoside)-4-ketopentanoic acid;
71. 5-(C-β-L-fucopyranoside) hexane-2,6-diol)-4-ketopentanoic acid;
72. 5-(C-α-L-fucopyranoside) hexane-2,6-diol)-4-ketopentanoic acid;
73. 5-(C-β-D-fucopyranoside)-4-hydroxypentanoic acid;
74. 5-(C-α-D-fucopyranoside)-4-hydroxypentanoic acid;
75. 5-(C-β-L-fucopyranoside)-4-hydroxypentanoic acid;
76. 5-(C-α-L-fucopyranoside)-4-hydroxypentanoic acid;
77. 5-(C-β-D-fucopyranoside)-4-aminopentanoic acid;
78. 5-(C-α-D-fucopyranoside)-4-aminopentanoic acid;
79. 5-(C-β-L-fucopyranoside)-4-aminopentanoic acid;
80. 5-(C-α-L-fucopyranoside)-4-aminopentanoic acid;
81. 1-(C-β-D-fucopyranoside) pentane-2,5-diol;
82. 1-(C-α-D-fucopyranoside) pentane-2,5-diol;
83. 1-(C-β-L-fucopyranoside) pentane-2,5-diol;
84. 1-(C-α-L-fucopyranoside) pentane-2,5-diol;
85. 1-(C-β-D-glucopyranosyl)-2-hydroxypropane;
86. 1-(C-α-D-glucopyranosyl)-2-hydroxypropane;
87. 1-(C-β-D-glucopyranosyl)-2-aminopropane;
88. 1-(C-α-D-glucopyranosyl)-2-aminopropane;
89. 1-(C-β-D-glucopyranosyl)-2-phenylaminopropane;
90. 1-(C-α-D-glucopyranosyl)-2-phenylaminopropane;
91. ethyl ester of 3-methyl-4-(C-β-D-glucopyranosyl)-butyric acid;
92. ethyl ester of 3-methyl-4-(C-α-D-glucopyranosyl)-butyric acid;
93. 6-(C-β-D-glucopyranosyl)-5-ketohexanoic acid;
94. 6-(C-α-D-glucopyranosyl)-5-ketohexanoic acid;
95. 6-(C-β-D-glucopyranosyl)-5-hydroxyhexanoic acid;
96. 6-(C-α-D-glucopyranosyl)-5-hydroxyhexanoic acid;
97. 6-(C-β-D-glucopyranosyl)-5-aminohexanoic acid;
98. 6-(C-α-D-glucopyranosyl)-5-aminohexanoic acid;
99. 6-(C-β-D-glucopyranosyl)-5-phenylaminohexanoic acid;
100. 6-(C-α-D-glucopyranosyl)-5-phenylaminohexanoic acid;
101. 1-(C-β-D-glucopyranosyl) hexane-2,6-diol;
102. 1-(C-α-D-glucopyranosyl) hexane-2,6-diol;
103. 6-(C-β-D-glucopyranosyl)-5-ketopentanoic acid;
104. 6-(C-α-D-glucopyranosyl)-5-ketopentanoic acid;
105. 6-(C-β-D-glucopyranosyl)-5-hydroxypentanoic acid;
106. 6-(C-α-D-glucopyranosyl)-5-hydroxypentanoic acid;
107. 6-(C-β-D-glucopyranosyl)-5-aminopentanoic acid;
108. 6-(C-α-D-glucopyranosyl)-5-hydroxypentanoic acid;
109. 6-(C-β-D-glucopyranosyl)-5-phenylaminopentanoic acid;
110. 6-(C-α-D-glucopyranosyl)-5-phenylaminopentanoic acid;
111. 1-(C-β-D-glucopyranosyl) pentane-2,5-diol;
112. 1-(C-α-D-glucopyranosyl) pentane-2,5-diol;
113. 1-(C-β-D-galactopyranosyl)-2-hydroxypropane;
114. 1-(C-α-D-galactopyranosyl)-2-hydroxypropane;
115. 1-(C-β-D-galactopyranosyl)-2-aminopropane;
116. 1-(C-α-D-galactopyranosyl)-2-aminopropane;
117. 1-(C-β-D-galactopyranosyl)-2-phenylaminopropane;
118. 1-(C-α-D-galactopyranosyl)-2-phenylaminopropane;
119. ethyl ester of 3-methyl-4-(β-D-galactopyranosyl)-butyric acid;
120. ethyl ester of 3-methyl-4-(α-D-galactopyranosyl)-butyric acid;
121. 6-(C-β-D-galactopyranosyl)-5-ketohexanoic acid;
122. 6-(C-α-D-galactopyranosyl)-5-ketohexanoic acid;
123. 6-(C-β-D-galactopyranosyl)-5-hydroxyhexanoic acid;
124. 6-(C-α-D-galactopyranosyl)-5-hydroxyhexanoic acid;
125. 6-(C-β-D-galactopyranosyl)-5-aminohexanoic acid;
126. 6-(C-α-D-galactopyranosyl)-5-aminohexanoic acid;
127. 6-(C-β-D-galactopyranosyl)-5-phenylaminohexanoic acid;
128. 6-(C-α-D-galactopyranosyl)-5-phenylaminohexanoic acid;
129. 1-(C-β-D-galactopyranosyl) hexane-2,6-diol;
130. 1-(C-α-D-galactopyranosyl) hexane-2,6-diol;
131. 6-(C-β-D-galactopyranosyl)-5-ketopentanoic acid;
132. 6-(C-α-D-galactopyranosyl)-5-ketopentanoic acid;
133. 6-(C-β-D-galactopyranosyl)-5-hydroxypentanoic acid;
134. 6-(C-α-D-galactopyranosyl)-5-hydroxypentanoic acid;
135. 6-(C-β-D-galactopyranosyl)-5-aminopentanoic acid;
136. 6-(C-α-D-galactopyranosyl)-5-aminopentanoic acid;
137. 6-(C-β-D-galactopyranosyl)-5-phenylaminopentanoic acid;
138. 6-(C-α-D-galactopyranosyl)-5-phenylaminopentanoic acid;
139. 1-(C-β-D-galactopyranosyl) pentane-2,6-diol;
140. 1-(C-α-D-galactopyranosyl) pentane-2,6-diol;
141. 1-(C-β-D-fucofuranosyl) propan-2-one;
142. 1-(C-α-D-fucofuranosyl) propan-2-one;
143. 1-(C-β-L-fucofuranosyl) propan-2-one;
144. 1-(C-α-L-fucofuranosyl) propan-2-one;
145. 3′-(acetamido-C-β-D-glucopyranosyl) propane-2′-one;
146. 3′-(acetamido-C-α-D-glucopyranosyl) propane-2′-one;
147. 1-(acetamido-C-β-D-glucopyranosyl)-2-hydroxypropane;
148. 1-(acetamido-C-β-D-glucopyranosyl)-2-aminopropane;
149. 1-(acetamido-C-β-D-glucopyranosyl)-2-phenylaminopropane;
150. 1-(acetamido-C-α-D-glucopyranosyl)-2-phenylaminopropane;
151. ethyl ester of 3-methyl-4-(acetamido-C-β-D-glucopyranosyl) butyric acid;
152. ethyl ester of 3-methyl-4-(acetamido-C-α-D-glucopyranosyl) butyric acid;
153. 6-(acetamido-C-β-D-glucopyranosyl)-5-ketohexanoic acid;
154. 6-(acetamido-C-α-D-glucopyranosyl)-5-ketohexanoic acid;
155. 6-(acetamido-C-β-D-glucopyranosyl)-5-hydroxyhexanoic acid;
156. 6-(acetamido-C-α-D-glucopyranosyl)-5-hydroxyhexanoic acid;
157. 6-(acetamido-C-β-D-glucopyranosyl)-5-aminohexanoic acid;
158. 6-(acetamido-C-α-D-glucopyranosyl)-5-aminohexanoic acid;
159. 6-(acetamido-C-β-D-glucopyranosyl)-5-phenylaminohexanoic acid;
160. 6-(acetamido-C-α-D-glucopyranosyl)-5-phenylaminohexanoic acid;
161. 1-(acetamido-C-β-D-glucopyranosyl) hexane-2,6-diol;
162. 1-(acetamido-C-α-D-glucopyranosyl) hexane-2,6-diol;
163. 6-(acetamido-C-β-D-glucopyranosyl)-5-ketopentanoic acid;
164. 6-(acetamido-C-α-D-glucopyranosyl)-5-ketopentanoic acid;
165. 6-(acetamido-C-β-D-glucopyranosyl)-5-hydroxypentanoic acid;
166. 6-(acetamido-C-α-D-glucopyranosyl)-5-hydroxypentanoic acid;
167. 6-(acetamido-C-β-D-glucopyranosyl)-5-aminopentanoic acid;
168. 6-(acetamido-C-α-D-glucopyranosyl)-5-aminopentanoic acid;
169. 6-(acetamido-C-β-D-glucopyranosyl)-5-phenylaminopentanoic acid;
170. 6-(acetamido-C-α-D-glucopyranosyl)-5-phenylaminopentanoic acid;
171. 1-(acetamido-C-β-D-glucopyranosyl) pentane-2,5-diol;
172. 1-(acetamido-C-α-D-glucopyranosyl) pentane-2,5-diol.

Even more preferentially, the C-glycosides used in accordance with the invention are chosen from:
C-β-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside) propan-2-one,
1-(C-α-D-fucopyranoside) propan-2-one,
1-(C-β-L-fucopyranoside) propan-2-one,
1-(C-α-L-fucopyranoside) propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-Glucopyranosyl)-2-hydroxypropane,
1-(C-α-D-Glucopyranosyl)-2-hydroxypropane,
1-(C-β-D-galactopyranosyl)-2-hydroxypropane,
1-(C-α-D-galactopyranosyl)-2-hydroxypropane
1-(C-β-D-fucofuranosyl) propan-2-one,
1-(C-α-D-fucofuranosyl) propan-2-one
1-(C-β-L-fucofuranosyl) propan-2-one,
1-(C-α-L-fucofuranosyl) propan-2-one,
C-β-D-maltopyranoside-n-propan-2-one,
C-α-D-maltopyranoside-n-propan-2-one
C-β-D-maltopyranoside-2-hydroxypropane,
C-α-D-maltopyranoside-2-hydroxypropane, isomers thereof or mixtures thereof.

Better still, the C-glycoside used in the context of the invention is C-β-D-xylopyranoside-2-hydroxypropane or C-α-D-xylopyranoside-2-hydroxypropane, and preferably C-β-D-xylopyranoside-2-hydroxypropane such as the product sold under the name Mexoryl SBB® or Mexoryl SCN® by Noveal, the INCI name of which is Hydroxypropyl Tetrahydropyrantriol or under the name Mexoryl SBB®, which contains 35% by weight of hydroxypropyl tetrahydropyrantriol in 40% by weight of water and 25% of propylene glycol.

Said C-glycoside may be present in the composition in an amount ranging from 0.001% to 10% by weight of active material relative to the total weight of the composition, preferably from 0.005% to 5% by weight of active material, more preferentially from 0.01% to 4% by weight of active material relative to the total weight of the composition, better still from 0.5% to 3.5% by weight of active material relative to the total weight of the composition, such as 0.6% or 3.2% by weight of active material relative to the total weight of the composition.

In a preferred embodiment, said C-glycoside may be present in the composition in an amount of at least 0.01% by weight, preferably at least 0.5% by weight of active material relative to the total weight of the composition.

The extract of the biomass of yeast of the species Aureobasidium pullulans may be present in the composition in a content ranging from 0.0005% to 1% by weight of dry matter, preferably ranging from 0.001% to 1% by weight of dry matter, more preferably ranging from 0.005% to 0.5% by weight of dry matter, and even better ranging from 0.01% to 0.3% by weight, relative to the total weight of the composition.

Preferably, the mass ratio [C-glycoside(s)/extract of the biomass of yeast of the species Aureobasidium pullulans] is less than or equal to 50, more preferentially the mass ratio [C-glycoside(s)/extract of the biomass of yeast of the species Aureobasidium pullulans] is between 5 and 50, better still between 8 and 49.

### Forms of the Compositions

The description of the compositions may apply both to the compositions according to the invention and to the compositions in which the cosmetic active principle comprising the extract may be formed in the context of the use which is the subject of the present invention. Such compositions may be in the form of cosmetic compositions for caring for keratin materials, preferably cosmetic compositions for caring for keratin materials, in particular the body or the face, preferably the face.

These compositions may constitute cleansing, protective, treatment or care creams for the face, for the hands, or for the body including the skin of the scalp, for example day creams, night creams, makeup creams, foundation creams, antisun creams or hair lotions or creams for the scalp.

In particular, the compositions may be in the form of anti-ageing care, moisturizing or photoprotective compositions, in particular anti-ageing care compositions, for the skin of the body or the face, in particular the face.

The compositions may be applied to the skin by hand or using an applicator. More generally, the compositions may be characterized as containing a physiologically acceptable medium, i.e. a medium that is suitable for administering a composition topically, i.e. a medium that is compatible with the skin.

According to the invention, a physiologically acceptable medium is preferentially a cosmetically acceptable medium, i.e. odourless and with no unpleasant appearance, and which is entirely compatible with the topical administration route, i.e. which has a pleasant colour and feel and does not generate any unacceptable discomfort, i.e. tingling, tautness or redness, liable to discourage the user from applying this composition.

The compositions according to the invention may be in the form of aqueous solutions, aqueous-alcoholic solutions, oil-in-water (O/W) emulsions, water-in-oil (W/O) emulsions or multiple (triple: O/W/O or W/O/W) emulsions or aqueous gels, a dispersion of oils in an aqueous phase, notably using spherules, these spherules possibly being polymeric particles or, better still, lipid vesicles of the ionic and/or nonionic type, or else in the form of a powder, a serum, a paste or a flexible wand or a stick. It may be of solid, pasty or more or less fluid liquid consistency. These compositions are prepared according to the usual methods.

### Fatty Phase

A composition may comprise at least one fatty phase.

The fatty phase preferably contains at least one oil, notably a cosmetic oil. It may also contain other fatty substances.

The term “oil” means a water-immiscible non-aqueous compound that is liquid at room temperature (20° C.) and at atmospheric pressure (760 mmHg).

A fatty phase that is suitable for preparing the compositions, notably cosmetic compositions, according to the invention may comprise hydrocarbon-based oils, silicone oils, fluoro oils or non-fluoro oils, or mixtures thereof.

Preferably, a composition comprises less than 5.0% by weight of silicone oil(s), more preferentially less than 2.0% by weight of silicone oil(s), better still less than 1% by weight, relative to the total weight of the composition, and more preferentially is free of silicone oil(s).

A composition comprising a limited content of silicone oil(s) is advantageously more natural, but also lighter, less tacky and less rough to the touch, with a softer finish, than a composition comprising more than 5% by weight or more of silicone oil(s), relative to the total weight of the composition.

The oils may be volatile or nonvolatile.

They may be of animal, plant, mineral or synthetic origin.

The term “non-volatile” refers to an oil whose vapour pressure at room temperature and atmospheric pressure is non-zero and is less than 10-3 mmHg (0.13 Pa).

For the purposes of the present invention, the term “silicone oil” means an oil comprising at least one silicon atom, and particularly at least one Si—O group.

The term “fluoro oil” means an oil comprising at least one fluorine atom.

The term “hydrocarbon-based oil” means an oil mainly containing hydrogen and carbon atoms and possibly one or more heteroatoms such as oxygen or nitrogen atoms, and not containing any silicon or fluorine atoms. It may thus contain alcohol, ester, ether, carboxylic acid, amine and/or amide groups.

The oils may optionally comprise oxygen, nitrogen, sulfur and/or phosphorus atoms, for example in the form of hydroxyl or acid radicals.

For the purposes of the invention, the term “volatile oil” refers to any oil that is capable of evaporating on contact with the skin in less than one hour, at room temperature and atmospheric pressure. The volatile oil is a volatile cosmetic compound, which is liquid at room temperature, notably having a non-zero vapour pressure, at room temperature and atmospheric pressure, notably having a vapour pressure ranging from 0.13 Pa to 40 000 Pa (10-3 to 300 mmHg), in particular ranging from 1.3 Pa to 13 000 Pa (0.01 to 100 mmHg) and more particularly ranging from 1.3 Pa to 1300 Pa (0.01 to 10 mmHg).

Mention may notably be made of volatile hydrocarbon-based oils containing from 8 to 16 carbon atoms, branched C₈-C₁₆ alkanes, for instance C₈-C₁₆ isoalkanes (also known as isoparaffins), isododecane, isodecane, isohexadecane and, for example, the oils sold under the trade names Isopars or Permethyls, branched C₈-C₁₆ esters, for instance isohexyl neopentanoate, and mixtures thereof. In particular, the volatile hydrocarbon-based oil is chosen from volatile hydrocarbon-based oils containing from 8 to 16 carbon atoms and mixtures thereof.

Mention may also be made of volatile linear alkanes comprising from 8 to 16 carbon atoms, in particular from 10 to 15 carbon atoms and more particularly from 11 to 13 carbon atoms, for instance n-dodecane (C₁₂) and n-tetradecane (C₁₄) sold by Sasol under the respective references Parafol® 12-97 and Parafol® 14-97, and also mixtures thereof, the undecane-tridecane mixture, mixtures of n-undecane (C₁₁) and of n-tridecane (C₁₃) obtained in examples 1 and 2 of patent application WO 2008/155059 from the company Cognis, and mixtures thereof.

The following mixtures of linear or branched alkanes, preferably of plant origin, may also be mentioned:
a mixture of C₁₅-C₁₉ branched alkanes, for example the product sold by the company SEPPIC under the name Emogreen® L15;
a mixture of C₁₅-C₁₉ linear and/or branched alkanes, for example the product sold by the company SEPPIC under the name Emogreen® L19.

A composition according to the invention may comprise at least one hydrocarbon-based oil chosen from volatile linear alkanes comprising from 11 to 13 carbon atoms, in particular an undecane-tridecane mixture, and linear and/or branched C₁₅-C₁₉ alkanes, in particular a mixture of linear and/or branched C₁₅-C₁₉ alkanes.

Volatile silicone oils that may be mentioned include linear volatile silicone oils such as hexamethyldisiloxane, octamethyltrisiloxane, decamethyltetrasiloxane, tetradecamethylhexasiloxane, hexadecamethylheptasiloxane and dodecamethylpentasiloxane.

Volatile cyclic silicone oils that may be mentioned include hexamethylcyclotrisiloxane, octamethylcyclotetrasiloxane, decamethylcyclopentasiloxane, cyclohexasiloxane and dodecamethylcyclohexasiloxane, in particular cyclohexasiloxane.

Mention may also be made of nonvolatile hydrocarbon-based, fluoro and/or silicone oils.

Non-volatile hydrocarbon-based oils that may notably be mentioned include:
hydrocarbon-based oils of animal origin, such as squalane;
hydrocarbon-based oils of mineral or synthetic origin, such as:
   petroleum jelly,
   liquid paraffins,
   polybutylenes, for example Indopol H-100 (molar mass or MW=965 g/mol), Indopol H-300 (MW=1340 g/mol) and Indopol H-1500 (MW=2160 g/mol) sold or manufactured by the company Amoco,
   polyisobutenes and hydrogenated polyisobutenes, for example Parléam® sold by the company Nippon Oil Fats, Panalane H-300 E sold or manufactured by the company Amoco (MW=1340 g/mol), Viseal 20000 sold or manufactured by the company Synteal (MW=6000 g/mol) and Rewopal PIB 1000 sold or manufactured by the company Witco (MW=1000 g/mol),
   polydecenes and hydrogenated polydecenes, for example Puresyn 10 (MW=723 g/mol) and Puresyn 150 (MW=9200 g/mol) sold or manufactured by the company Mobil Chemicals; decene/butene copolymers,
   polybutene/polyisobutene copolymers, for example Indopol L-14,
hydrocarbon-based oils of plant origin, such as vegetable squalane,
synthetic ethers containing from 10 to 40 carbon atoms, such as dicaprylyl ether,
synthetic esters, for instance the oils of formula R₁COOR₂, in which R₁ represents a linear or branched fatty acid residue including from 1 to 40 carbon atoms and R₂ represents a hydrocarbon-based chain that is notably branched, containing from 1 to 40 carbon atoms, on condition that R₁+R₂ is greater than or equal to 10. The esters may notably be chosen from esters of alcohol and of fatty acid, for instance cetostearyl octanoate, esters of isopropyl alcohol, such as isopropyl myristate, isopropyl palmitate, ethyl palmitate, 2-ethylhexyl palmitate, isopropyl stearate, octyl stearate, hydroxylated esters, for instance isostearyl lactate, octyl hydroxystearate, alcohol or polyalcohol ricinoleates, hexyl laurate, neopentanoic acid esters, for instance isodecyl neopentanoate, isotridecyl neopentanoate, isononanoic acid esters, for instance isononyl isononanoate, isotridecyl isononanoate, octyl isononanoate, oleyl erucate, isopropyl lauroyl sarcosinate, diisopropyl sebacate, isocetyl stearate, isodecyl neopentanoate or isostearyl behenate;
polyol esters and pentaerythritol esters, for instance dipentaerythrityl tetrahydroxystearate/tetraisostearate,
fatty alcohols that are liquid at room temperature, with a branched and/or unsaturated carbon-based chain containing from 12 to 26 carbon atoms, for instance 2-octyldodecanol, isostearyl alcohol and oleyl alcohol,
C₁₂-C₂₂ higher fatty acids, such as oleic acid, linoleic acid, linolenic acid, and mixtures thereof,
carbonates, such as dicaprylyl carbonate,
non-phenyl silicone oils, for instance caprylyl methicone, and phenyl silicone oils, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates, dimethicones or phenyl trimethicone with a viscosity of less than or equal to 100 cSt, trimethylpentaphenyltrisiloxane, and mixtures thereof;
and also mixtures of these various oils.

The oil(s) may be present in a composition according to the invention in a content ranging from 0.1% to 20% by weight and preferably from 3% to 15% by weight relative to the total weight of the composition.

The composition according to the invention may also comprise solid fatty substances, for instance fatty acids that are solid at room temperature, such as stearic acid, lauric acid and palmitic acid; waxes, such as lanolin, beeswax, carnauba wax or candelilla wax, paraffin wax, lignite wax or microcrystalline wax, ceresin or ozokerite, synthetic waxes such as polyethylene waxes, Fischer-Tropsch waxes, fatty alcohol waxes, butters such as plant butters; silicone resins such as trifluoromethyl-C₁-C₄-alkyldimethicone and trifluoropropyldimethicone; and silicone elastomers such as the products sold under the name KSG by the company Shin-Etsu, under the name Trefil or BY29 by the company Dow Corning or under the name Gransil by the company Grant Industries.

As fatty alcohol waxes, mention may be made of lauryl alcohol, myristyl alcohol, cetyl alcohol, stearyl alcohol, arachidyl alcohol, behenyl alcohol, lignoceryl alcohol, ceryl alcohol, montanyl alcohol, myricyl alcohol, and mixtures thereof.

Preferentially, the fatty alcohol wax is cetyl alcohol.

As butters, in particular plant butters, mention may be made of avocado butter, cocoa butter, shea butter, mango butter, coconut butter, apricot kernel butter and sal butter, and mixtures thereof, and in particular is shea butter.

These fatty substances may be chosen in a varied manner by a person skilled in the art in order to prepare a composition having the desired properties, for example in terms of consistency or texture.

The solid fatty substance(s) may be present in a composition according to the invention in a content ranging from 0.1% to 10% by weight and preferably from 0.5% to 5% by weight, relative to the total weight of the composition.

### Aqueous Phase

A composition may comprise at least one aqueous phase.

The aqueous phase may comprise water and optionally a water-miscible organic solvent.

The water used may be demineralized water and/or a floral water such as rose water, cornflower water, chamomile water or lime blossom water, and/or a natural spring water or mineral water, for instance: Vittel water, Vichy basin water, Uriage water, Roche Posay water, Bourboule water, Enghien-les-Bains water, Saint Gervais-les-Bains water, Néris-les-Bains water, Allevar-les-Bains water, Digne water, Maizières water, Neyrac-les-Bains water, Lons-le-saunier water, Eaux Bonnes water, Rochefort water, Saint Christau water, Fumades water, Tercis-les-Bains water and Avene water. The aqueous phase may also comprise reconstituted spring water, i.e. a water containing trace elements such as zinc, copper, magnesium, etc., reconstituting the characteristics of a spring water.

The water-miscible organic solvents that may be used in the composition of the invention may also be volatile.

The composition may comprise water in a concentration ranging from 20% to 95% by weight, relative to the total weight of the composition.

Preferably, water is present in a composition according to the invention in a content ranging from 30% to 95% by weight, preferably from 40% to 90% by weight, and more preferentially from 45% to 85% by weight, relative to the total weight of said composition.

According to the present invention, the term “water-miscible organic solvent” denotes an organic compound that is liquid at room temperature, having a water miscibility of greater than 50% by weight at 25° C. and atmospheric pressure.

Among the water-miscible organic solvents that may be used in the composition according to the invention, mention may notably be made of lower monoalcohols containing from 1 to 5 carbon atoms, polyols, C₃ and C₄ ketones and C₂-C₄ aldehydes.

Among the lower monoalcohols containing from 1 to 5 carbon atoms, mention may be made of ethanol and isopropanol.

The term “polyol that is suitable for use in the invention” means a compound of linear, branched or cyclic, saturated or unsaturated alkyl type, bearing on the alkyl chain at least two —OH functions, in particular at least three-OH functions and more particularly at least four —OH functions.

The polyols that are suitable for formulating a composition according to the present invention are in particular those notably containing from 2 to 32 carbon atoms and preferably 3 to 16 carbon atoms.

Among the polyols, mention may be made of pentaerythritol, trimethylolpropane, ethylene glycol, hexylene glycol, propylene glycol, 1,3-butylene glycol, isoprene glycol, pentylene glycol, caprylyl glycol, dipropylene glycol, glycerol, polyglycerols, such as glycerol oligomers, for instance diglycerol, and polyethylene glycols.

When they are present, the water-miscible organic solvent(s) are preferably present in a composition according to the invention in a content ranging from 1% to 20% by weight, better still from 3% to 15% by weight, preferably from 5% to 15% by weight, relative to the total weight of said composition.

The aqueous phase may also comprise any water-soluble or water-dispersible compound that is compatible with an aqueous phase, such as gelling agents, film-forming polymers, thickeners or surfactants, and mixtures thereof.

Depending on the viscosity of the composition that it is desired to obtain, one or more thickeners and/or gelling agents, which are notably hydrophilic, that is to say water-soluble or water-dispersible, may be incorporated into the composition.

Advantageously, the gelling agent is chosen from hydrophilic polymeric gelling agents that are synthetic or natural or of natural origin, and mixtures thereof.

For the purposes of the invention, the term “of natural origin” is intended to denote polymeric gelling agents obtained by modification of natural polymeric gelling agents. The synthetic polymeric hydrophilic gelling agents may be chosen from crosslinked acrylic homopolymers or copolymers; associative polymers, in particular associative polymers of polyurethane type; polyacrylamides and optionally crosslinked and/or neutralized 2-acrylamido-2-methylpropanesulfonic acid polymers and copolymers; modified or unmodified carboxyvinyl polymers, and mixtures thereof, notably as defined below.

Among the crosslinked acrylic homopolymers or copolymers, mention may be made of crosslinked sodium polyacrylates, for instance those sold under the brand names Octacare X100, X110 and RM100 by the company Avecia, those sold under the names Flocare GB300 and Flosorb 500 by the company SNF, those sold under the names Luquasorb 1003, Luquasorb 1010, Luquasorb 1280 and Luquasorb 1110 by the company BASF, those sold under the names Water Lock G400 and G430 (INCI name: Acrylamide/Sodium acrylate copolymer) by the company Grain Processing.

Among the carboxyvinyl polymers, examples that may be mentioned include modified or unmodified carboxyvinyl polymers, such as the products sold under the name Carbopol® (CTFA name: carbomer).

Among the polyacrylamides and the optionally crosslinked and/or neutralized 2-acrylamido-2-methylpropanesulfonic acid polymers and copolymers, mention may be made of poly(2-acrylamido-2-methylpropanesulfonic acid) sold by the company Hoechst under the name Hostacerin® AMPS (CTFA name: Ammonium polyacryldimethyltauramide), crosslinked anionic copolymers of acrylamide and of AMPS, which are in the form of a water-in-oil emulsion, such as those sold under the name Sepigel® 305 (CTFA name: Polyacrylamide/C₁₃₋₁₄ Isoparaffin/Laureth-7) and under the name Simulgel® 600 (CTFA name: Acrylamide/sodium acryloyldimethyltaurate copolymer/isohexadecane/polysorbate 80) by the company SEPPIC.

The hydrophilic polymeric gelling agents that are natural or of natural origin may be chosen from modified or unmodified celluloses, carrageenans, gellan gum, agar-agar, xanthan gum, alginate-based compounds, in particular sodium alginate, scleroglucan gum, guar gum, pullulan, cassia gum, karaya gum, konjac gum, gum tragacanth, tara gum, acacia gum or gum arabic, and mixtures thereof.

### Active Agents

Advantageously, a composition according to the invention may also comprise at least one additional cosmetic active agent.

Examples of active agents that may be mentioned include moisturizers, depigmenting agents, desquamating agents, humectants, anti-ageing agents, cicatrizing agents, and mixtures thereof.

### Surfactants

A composition according to the invention may comprise emulsifying surfactants, which are preferably nonionic.

The nonionic surfactants may notably be chosen from alkyl and polyalkyl esters of poly(ethylene oxide), oxyalkylenated alcohols, alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, in particular alkyl and polyalkyl glucosides or polyglucosides, alkyl and polyalkyl esters of sucrose, optionally polyoxyethylenated alkyl and polyalkyl esters of glycerol, and optionally polyoxyethylenated alkyl and polyalkyl ethers of glycerol, gemini surfactants, cetyl alcohol, stearyl alcohol, and mixtures thereof.

Oxyalkylenated, in particular oxyethylenated and/or oxypropylenated, alcohols that are preferably used are those that may include from 1 to 150 oxyethylene and/or oxypropylene units, in particular containing from 20 to 100 oxyethylene units, in particular fatty alcohols, notably of C₈-C₂₄ and preferably of C₁₂-C₁₈; these fatty alcohols may or may not be ethoxylated, for instance stearyl alcohol ethoxylated with 20 oxyethylene units (CTFA name Steareth-20), for instance Brij® 78 sold by the company Uniqema, cetearyl alcohol ethoxylated with 30 oxyethylene units (CTFA name Ceteareth-30), and the mixture of C₁₂-C₁₅ fatty alcohols including 7 oxyethylene units (CTFA name C₁₂-15 Pareth-7), for instance the product sold under the name Neodol 25-7® by Shell Chemicals; or in particular oxyalkylenated (oxyethylenated and/or oxypropylenated) alcohols containing from 1 to 15 oxyethylene and/or oxypropylene units, in particular ethoxylated C₈-C₂₄ and preferably C₁₂-C₁₈ fatty alcohols, such as stearyl alcohol ethoxylated with 2 oxyethylene units (CTFA name Steareth-2), for instance Brij® 72 sold by the company Uniqema.

Optionally polyoxyethylenated alkyl and polyalkyl esters of sorbitan that are preferably used include those with a number of ethylene oxide (EO) units ranging from 0 to 100. Examples that may be mentioned include sorbitan laurate 4 or 20 EO, in particular polysorbate 20 (or polyoxyethylene (20) sorbitan monolaurate) such as the product Tween® 20 sold by the company Uniqema, or polysorbate 60, sorbitan palmitate 20 EO, sorbitan isostearate, sorbitan stearate 20 EO, sorbitan oleate 20 EO, or else the Cremophor® products (RH 40, RH 60, etc.) from BASF. The mixture of sorbitan stearate and of sucrose cocoate, sold under the name Arlacel® 2121U-FL from Croda, may also be mentioned.

Alkyl and polyalkyl glucosides or polyglucosides that are preferably used are those containing an alkyl group including from 6 to 30 carbon atoms and preferably from 6 to 18 or even from 8 to 16 carbon atoms, and containing a glucoside group preferably comprising from 1 to 5 and notably 1, 2 or 3 glucoside units. The alkylpolyglucosides may be chosen, for example, from decylglucoside (alkyl-C₉/C₁₁-polyglucoside (1.4)), for instance the product sold under the name Mydol 10® by the company Kao Chemicals or the product sold under the name Plantacare 2000 UP® by the company Henkel and the product sold under the name Oramix NS 10® by the company SEPPIC; caprylyl/capryl glucoside, for instance the product sold under the name Plantacare KE 3711® by the company Cognis or Oramix CG 110® by the company SEPPIC; laurylglucoside, for instance the product sold under the name Plantacare 1200 UP® by the company Henkel or Plantaren 1200 N® by the company Henkel; cocoyl glucoside, for instance the product sold under the name Plantacare 818 UP® by the company Henkel; caprylyl glucoside, for instance the product sold under the name Plantacare 810 UP® by the company Cognis; the mixture of arachidyl glucosyl and behenyl alcohol and arachidyl alcohol, the INCI name of which is Arachidyl alcohol (and) behenyl alcohol (and) arachidyl glucoside, sold under the name Montanov® 202 by the company SEPPIC; the mixture of cetearyl alcohol and cetearyl glucoside, whose INCI name is Cetearyl alcohol/cetearyl glucoside sold under the name Montanov® 68 by the company SEPPIC, and mixtures thereof.

A composition according to the invention or as used according to the invention may comprise between 0.1% and 30% by weight of emulsifying surfactant, preferably between 0.2% and 20% by weight, more preferentially between 0.5% and 10% by weight, relative to the total weight of the composition.

The expressions “between . . . and . . . ”, “comprises from . . . to . . . ”, “formed from . . . to . . . ” and “ranging from . . . to . . . ” should be understood as being inclusive of the limits, unless otherwise specified.

The invention is illustrated in greater detail by the examples presented below. Unless otherwise indicated, the amounts indicated are expressed as mass percentages.

### EXAMPLES

### Example 1: Cosmetic Active Principle in Accordance with the Invention

The active principle of Example 1 is obtained from Aureobasidium pullulans yeast isolated from rose bushes. The active principle of Example 1 is obtained via the following process:
a. Cultivation of Aureobasidium pullulans in a suitable culture medium
b. Dissolution of the biomass of the Aureobasidium pullulans yeast in water at a rate of 50 g/l,
c. Extraction of the sugars,
d. Heat treatment between 80° C. and 90° C.,
e. Separation of the soluble and insoluble phases,
f. Purification by molecular sorting,
g. Concentration, and
h. Sterilizing filtration.

The active principle obtained has the following characteristics:
Dry matter content=20.1 g/L, of which:
Total Sugar Content (according to the Dubois method)=6.9 g/L (34%, by weight relative to the dry matter)
   i. Content of alpha-linked glucose oligosaccharides=2.5 g/L
   ii. Content of beta-linked glucose oligosaccharides=2.5 g/L
Peptide content (according to the Kjeldhal method)=6.6 g/L (33%, by weight relative to the dry matter)
Mineral ash content=2.5 g/L (12%, by weight relative to the dry matter)
pH=3.5
Clear, very pale yellow liquid, faint odour

### Example 2: Active Principle Outside the Invention

The active principle of Example 2 is also obtained from Aureobasidium pullulans yeast isolated from rose bushes. The active principle of Example 2 is obtained via the following process:
a. Cultivation of Aureobasidium pullulans in a suitable culture medium
b. Dissolution of the biomass of the Aureobasidium pullulans yeast in water at a rate of 50 g/l,
c. Extraction of the proteins,
d. Heat treatment between 80° C. and 90° C.,
e. Separation of the soluble and insoluble phases,
f. Purification by molecular sorting,
g. Concentration, and
h. Sterilizing filtration.

The active principle obtained has the following characteristics:
Dry matter content=26.8 g/L, of which:
Total Sugar Content (according to the Dubois method)=2.5 g/L (9%, by weight relative to the dry matter)
Peptide content (according to the Kjeldhal method)=12.4 g/L (46%, by weight relative to the dry matter)
Mineral ash content=5 g/L (19%, by weight relative to the dry matter)
pH=3.6
Clear, very pale yellow liquid, faint odour

### Example 3: Active Principle Outside the Invention

The active principle of Example 3 is obtained from the culture supernatant of the Aureobasidium pullulans biomass.
a. Cultivation of Aureobasidium pullulans in a suitable culture medium
b. Physical separation in order to recover the culture supernatant from the biomass
c. Sterilizing filtration.

The active principle obtained has the following characteristics:
Dry matter content=9.6 g/L, of which:
Total Sugar Content (according to the Dubois method)=1.6 g/L (17%, by weight relative to the dry matter)
Protein content (according to the Lowry method)=0.11 g/L (1%, by weight relative to the dry matter)
Mineral ash content=6.2 g/L (65%, by weight relative to the dry matter)
pH=6.9
Clear, pale yellow liquid, faint odour.

### Example 4: Effect of the Active Principle in Accordance with the Invention on the Ageing of Epidermal Compartment Cells

The present study is directed towards studying the expression of certain genes and determining the content of certain proteins in keratinocytes treated with the various extracts. The keratinocytes are aged using a UVB free radical stress, or are derived from elderly donors.

Table 1 lists the epidermal compartment genes modulated during ageing by free radical stress.

TABLE 1

| Symbol | Name |
|---|---|
| HSP90AA1 | Heat shock protein 90 alpha family class A member 1 |
| SIRT1 | Sirtuin 1 |

Table 2 lists the epidermal compartment proteins modulated during ageing by free radical stress.

TABLE 2

| Name | Symbol |
|---|---|
| Parkinson disease protein 7 | DJ-1 |
| Tumor necrosis factor receptor | TNR6 |
| superfamily member 6 | |

The results of the study are presented in Tables 3 to 5 below. Table 3 represents the expression of the genes HSP90AA1 and SIRT1 in keratinocytes subjected to a UVB free radical stress and treated with the various extracts.

TABLE 3

| | | | |
|---|---|---|---|
| HAP90AA1 | Efficacity | SIRT1 | Efficacity |
| (%) | (%) | (%) | (%) |

Non-aged, non-stressed keratinocytes

| | | |
|---|---|---|
| Control | 100 | 100 |

Keratinocytes aged by UVB stress

| | | | | |
|---|---|---|---|---|
| Control | 39 | | 16 | |
| EXAMPLE | 58 | +48 | 33 | +106 |
| 1 0.5% | | | | |
| EXAMPLE | 46 | +18 | 20 | +25 |
| 2 0.5% | | | | |
| EXAMPLE | 44 | +13 | 3 | 0 |
| 3 0.5% | | | | |

The sugar-rich active principle in accordance with the invention (Example 1) has a better effect on the expression of the genes HSP90AA1 and SIRT1 in keratinocytes aged by UVB stress than the extracts outside the invention (Example 2 and Example 3).

Table 4 below represents the rates of expression of the proteins DJ-1 and TNR6 in keratinocytes aged by UVB stress and treated with the various extracts.

TABLE 4

| | | | |
|---|---|---|---|
| DJ-1 | Efficacity | TNR6 | Efficacity |
| (%) | (%) | (%) | (%) |

Non-aged, non-stressed keratinocytes

| | | |
|---|---|---|
| Control | 100 | 100 |

Keratinocytes aged by UVB stress

| | | | | |
|---|---|---|---|---|
| Control | 2496 | | 507 | |
| EXAMPLE | 2227 | −11 | 310 | −39 |
| 1 0.5% | | | | |
| EXAMPLE | 2290 | −8 | 417 | −18 |
| 2 0.5% | | | | |
| EXAMPLE | 2453 | −2 | 507 | 0 |
| 3 0.5% | | | | |

The sugar-rich active principle in accordance with the invention (example 1) has a better effect on the rate of the proteins DJ-1 and TNR6 in keratinocytes aged by UVB stress than extracts not in accordance with the invention (Example 2 and Example 3).

Table 5 below represents the rates of expression of the protein DJ-1 in keratinocytes derived from elderly donors and treated with the active principle in accordance with the invention (Example 1).

TABLE 5

| | DJ-1 (%) | Efficacity (%) |
|---|---|---|
| Young keratinocytes | | |
| Control | 100 | |
| Aged keratinocytes | | |
| Control | 118 | |
| EXAMPLE 1 0.5% | 54 | −54 |

The sugar-rich active principle (Example 1) according to the invention has a significant effect on the rate of expression of the protein DJ-1 in keratinocytes from elderly donors. This result confirms the results obtained on keratinocytes aged by free radical stress. Thus, the sugar-rich extract according to Example 1 has an effect on the metabolic pathways of ageing (HSP90AA1+48%, SIRT1+106%, DJ 1-54%, TNR6-39%).

The protein-rich extract according to Example 2 has a much weaker effect on the metabolic pathways of ageing. It is thus less effective than the extract according to Example 1. The extract according to Example 3, extracted from the supernatant, has no effect on the metabolic pathways of ageing in the epidermal compartment.

Thus, a sugar-rich extract affords an improved effect on the metabolic pathways of ageing.

### Example 5: Effect of the Active Principle in Accordance with the Invention on the Migration of Aged Keratinocytes

The aim of this study is to investigate the ability of the various extracts to increase cell migration of normal human keratinocytes. Cell migration is the ability of certain cells to move, and this migratory capacity is reduced with age.

This study was performed on keratinocytes from elderly donors (>60 years old). Table 6 below represents the quantification of keratinocyte migration in the wound performed in cultures of aged keratinocytes, treated with the various extracts.

TABLE 6

| | Migration/ | Efficacity/ |
|---|---|---|
| | Control (%) | Control (%) |
| Control | 100 | |
| EXAMPLE 1 0.5% | 123 | +23 |
| EXAMPLE 3 0.5% | 100 | 0 |

The extract according to Example 3 has no effect on the migration of aged keratinocytes. In contrast, the extract according to Example 1 stimulated the migration of aged keratinocytes by 23%. In this case also, the sugar-rich extract according to the invention in Example 1 allows an improved effect in stimulating the migration of aged keratinocytes and thus anti-ageing efficacy.

### Example 6: In Vitro Effect of the Active Principle in Accordance with the Invention on Skin Barrier Function Markers

### A. Materials and Methods

### Culture and Treatment of Reconstructed Skins

The extract of Aureobasidium pullulans is that prepared in Example 1 above.

Human keratinocytes were seeded onto inserts and then incubated at 37° C. in an atmosphere containing 5% CO₂. Between the second and ninth day of incubation, the culture medium was changed every two days. Reconstructed epidermis samples were then treated systemically with either 0.25% and 0.50% (V/V) Aureobasidium pullulans extract or 250 μm niacinamide, and then incubated at 37° C. in an atmosphere containing 5% CO₂ from day 9 to day 17.

On day 17, the reconstructed skins were then recovered and processed in different ways depending on the analysis test performed, namely:
for immunohistofluorescence analysis, the reconstructed skins were recovered, fixed, dehydrated and embedded in paraffin. 4 μm sections were then produced using a microtome (Leica).
for a QPCR test, the reconstructed skins were recovered and the RNAs were extracted in their entirety.

### Analysis of Filaggrin, Loricin and Claudin-1 Synthesis by Immunohistofluorescence

The following primary antibodies were used for this test:
Mouse anti-filaggrin monoclonal antibody
Rabbit anti-loricrin polyclonal antibody
Mouse anti-claudin-1 monoclonal antibody

The secondary antibodies used were as follows:
Alexa Fluor® 488-coupled rabbit anti-IgG antibody
Alexa Fluor® 488-coupled mouse anti-IgG antibody

Visualization was performed using an IX 70 microscope (Olympus) coupled to an image analysis system (NIS-Elements software, Nikon). The content of the various markers synthesized was proportional to the intensity of the green fluorescence present on the reconstructed epidermis samples. Quantification was performed by means of an image analysis script in Python language. The results are expressed in arbitrary units (AU).

### Quantitative PCR Analysis of ZO-1, CK10, Involucrin and TGM1 Expression

For this analysis, the RNAs were reverse-transcribed and the complementary DNAs obtained were analysed via a quantitative PCR technique. The mRNAs of the RPS18, GAPDH and GUSB proteins, the reference internal controls, were analysed in parallel with the mRNAs of ZO-1, CK10, involucrin and TGM1.

Quantification of fluorescence incorporation (SYBR Green) was measured continuously using a LightCycler LC480 thermal cycler (Roche) and Ct analysis (relative quantification) was performed using the LC480 software (Roche).

### B. Results

*: significant results according to Student's t-test/control RECONSTRUCTED EPIDERMIS (p<0.05)
**: significant result according to Student's t-test/control RECONSTRUCTED EPIDERMIS (p<0.01)
***: significant results according to Student's t-test/control RECONSTRUCTED EPIDERMIS (p<0.005)
   Immunohistofluorescence Studies on the Effect of Aureobasidium pullulans Extract on the Synthesis of Epidermal Markers: Filaggrin, Loricrin and Claudin-1

TABLE 7

| | Synthesis | | Synthesis | | Synthesis | |
|---|---|---|---|---|---|---|
| | of filaggrin | Efficacy/ | of loricin | Efficacy/ | of claudin-1 | Efficacy/ |
| | (× 10³ AU) | control (%) | (× 10³ AU) | control (%) | (× 10³ AU) | control (%) |
| Control | 664 | — | 250 | — | 241 | — |
| Aureobasidium | 1221*** | +84 | 286* | +14 | 292* | +21 |
| pullulans extract | | | | | | |
| of Example 1 at | | | | | | |
| 0.25% | | | | | | |
| Aureobasidium | 1440*** | +117 | 310* | +24 | 335*** | +39 |
| pullulans extract | | | | | | |
| of Example 1* at | | | | | | |
| 0.50% | | | | | | |
| Niacinamide | 941 | +42 | 214 | — | 266 | +10 |
| 250 μm | | | | | | |

*the Aureobasidium pullulans extract of Example 1 was tested in the form of a mixture comprising 2.15% by weight of dry matter of Aureobasidium pullulans extract, 15% by weight of propylene glycol, qs water.

The study of the effect of the Aureobasidium pullulans extract on the synthesis of the epidermal markers filaggrin, loricrin and claudin-1 showed significant results relative to the control. For example, the effect of the Aureobasidium pullulans extract on the synthesis of claudin-1 was significant relative to the control.

### QPCR Studies

TABLE 8

| | | | | | | | Expression | |
|---|---|---|---|---|---|---|---|---|
| | Expression | Efficacy/ | Expression | Efficacy/ | Expression | Efficacy/ | of | Efficacy/ |
| | of ZO-1 | control | of CK10 | control | of TGMI | control | involucrin | control |
| | (%) | (%) | (%) | (%) | (%) | (%) | (%) | (%) |
| Control | 100 | — | 100 | — | 100 | — | 100 | — |
| Aureobasidium | 127* | +27 | 125* | +25 | 123* | +23 | 127* | +27 |
| pullulans extract | | | | | | | | |
| of Example 1* | | | | | | | | |
| at 0.50% | | | | | | | | |
| Niacinamide | 91 | — | 100 | — | 86 | — | 78 | — |
| 250 μm | | | | | | | | |

*the Aureobasidium pullulans extract of Example 1 was tested in the form of a mixture comprising 2.15% by weight of dry matter of Aureobasidium pullulans extract, 15% by weight of propylene glycol, qs water.

The study of the effect of the Aureobasidium pullulans extract on the synthesis of the epidermal markers ZO-1, CK10, TGM1 and involucrin showed significant results relative to the control. For example, the effect of the Aureobasidium pullulans extract on the expression of CK10 or involucrin was significant relative to the control.

As a result, the Aureobasidium pullulans extract allows the skin barrier function to be improved.

### Example 7: In Vivo Effect of the Active Principle in Accordance with the Invention on the Skin Barrier Function

### A. Materials and Methods

The extract of Aureobasidium pullulans is that prepared in Example 1 above.

The composition according to the invention, according to table 9 below, named “test composition” below, and also a second placebo formula identical to the “test composition” without the Aureobasidium pullulans extract, were applied to the half-face according to a predefined randomization for 42 days, by light massages until penetration of the composition.

The values are expressed as weight percentages of active material, relative to the total weight of the composition.

TABLE 9

| Ingredients | Percentage amount |
|---|---|
| Isononyl isononanoate (Massocare ININ, | 5.0% |
| CQ MASSO) | |
| Behenyl alcohol/arachidyl glucoside/ | 3.0% |
| arachidyl alcohol (Montanov 202, SEPPIC) | |
| Aureobasidium pullulans extract* | 2.5% |
| Cetearyl alcohol/cetearyl glucoside | 2.0% |
| (Montanov 68, SEPPIC) | |
| Preserving agents | 1.0% |
| Polyacrylamide/C13-14 isoparaffin/ | 0.3% |
| laureth 7 (Sepigel 305, SEPPIC) | |
| Citric acid | Qs pH 5 |
| Water | Qs 100% |

*the Aureobasidium pullulans extract of Example 1 was tested in the form of a mixture comprising 2.15% by weight of dry matter of Aureobasidium pullulans extract, 15% by weight of propylene glycol, qs water.

The panel of users (20 volunteers) underwent a “wash-out” period for 14 days, with twice-daily application of the placebo formula. Then, every morning and evening, the users applied a composition containing the Aureobasidium pullulans extract and also the placebo formula per half-face to their skin, free of cream and/or makeup, for a period of 42 days. On the day of the measurements, the volunteers came to the laboratory without having applied any product to the areas of interest in the morning (neither cream nor makeup).

### Study of the Barrier Function

The measurements of the transepidermal water loss (TEWL) were performed using a Tewameter® TM 300 (Courage&Khazaka), comprising a probe which measures the water vapour gradient between the skin surface and the ambient air, which gives information regarding the quality of the barrier function of the stratum corneum.

Said TEWL measurements were performed on symmetrical areas of the cheeks at different times during the study.

A reduction in the TEWL is characteristic of an improvement in the skin's barrier function. The effect of the Aureobasidium pullulans extract on the TEWL was measured on the face compared with placebo after 14, 28 and 42 days of twice-daily application per half-face.

### Study of Skin Microrelief

The study of skin microrelief was conducted using impressions analysed by fringe projection.

These silicone polymer impressions were performed on the cheeks at various stages of the study.

Volume acquisitions of these impressions were then performed using a fringe projection apparatus dedicated to the 3D measurement of impression relief (Eotech). This system (DermaTOP 1303) comprises a measurement sensor combining a projector and a high-resolution CCD camera linked to Optocat acquisition software (Eotech). The area under study is cut out automatically from the original acquisition.

The parameter chosen for this study is the parameter Sa (mm), which corresponds to the arithmetic mean of the surface roughness.

The higher this parameter, the rougher the surface. A reduction in this parameter is characteristic of an improvement in microrelief.

### B. Data Processing

The effect of the Aureobasidium pullulans extract and the placebo was evaluated by studying the degree of change between DO and the different times of interest (Dx), reflecting the variation between before and after treatment.

$$Δ / D 0 ( \% ) = \frac{( M {V}_{Dx} - {MV}_{D 0} )}{{MV}_{J 0}} \qquad [ formula 1 ]$$

with:
MV_{Dx}: Mean value on the treated area before treatment
MVⱼ₀: Mean value on the treated area after x days of daily application

The effect of the Aureobasidium pullulans extract relative to the placebo formula was evaluated by taking the difference between the degree of change for the formula containing the Aureobasidium pullulans extract and that for the placebo formula:

$$Δ / Placebo ( \% ) = {( Δ / D 0 )}_{Product} - {( Δ / D 0 )}_{Placebo} \qquad [ formula 2 ]$$

with:
(Δ/D0)_{Product}: degree of change relative to D0 in the area treated with the test composition comprising Aureobasidium pullulans extract after x days of daily applications.
(Δ/D0)_{Placebo}: degree of change relative to DO on the area treated with the placebo after x days of daily applications.

### Statistical Data Analysis

Comparisons of the results obtained between DO and the various times of interest, after using each of the formulas and between the effect of the formula containing the Aureobasidium pullulans extract and that of the placebo formula were performed using Student's t-test on paired data, except when the result of the Shapiro-Wilk normality test was less than 5%. In this case, statistical analysis was performed using the non-parametric Wilcoxon Signed Ranks test. In both cases, the test is one-tailed with a risk threshold set at 5%. The Statgraphics™ Centurion version XVI software was used to perform these analyses.

### C. Results

Study of the barrier function: Effect of the Aureobasidium pullulans extract on the TEWL (g/h/m²) measured on the face compared with placebo after 14, 28 and 42 days of twice-daily application.

TABLE 10

| | | | | | Test composition comprising the | | | |
|---|---|---|---|---|---|---|---|---|
| | Placebo | | | | Aureobasidium pullulans extract | | | |
| Volunteers | D0 | D14 | D28 | D42 | D0 | D14 | D28 | D42 |
| Mean | 8.47 | 8.95 | 8.83 | 8.73 | 8.84 | 8.04 | 7.86 | 7.74 |
| SEM | 0.52 | 0.45 | 0.39 | 0.38 | 0.52 | 0.35 | 0.42 | 0.50 |
| A/D0 (%) | — | +5.6 | +4.3 | +3.1 | — | −9.0* | −11.1* | −12.4* |
| p-value | — | 0.8391 | 0.6214 | 0.5913 | — | 0.0324 | 0.0189 | 0.0259 |

*significant results/D0 according to Student's t-test (p < 0.05)

In comparison with placebo, the formula containing the Aureobasidium pullulans extract as an emulsion showed an improvement in the quality of the barrier function from 14 days of treatment (−14.6%, p=0.0026). This effect continued after 28 and 42 days of treatment with −15.4% (p=0.0066) and −15.5% (p=0.0004) respectively, and was observed in more than 75% of volunteers at the different measurement times).

Study of skin microrelief: Smoothing effect of the Aureobasidium pullulans extract on cheek microrelief, parameter Sa (mm) in comparison with placebo after 14, 28 and 42 days of twice-daily application.

TABLE 11

| | | | | | Test composition comprising the | | | |
|---|---|---|---|---|---|---|---|---|
| | Placebo | | | | Aureobasidium pullulans extract | | | |
| Volunteers | D0 | D14 | D28 | D42 | D0 | D14 | D28 | D42 |
| Mean | 0.027 | 0.027 | 0.029 | 0.028 | 0.026 | 0.023 | 0.026 | 0.025 |
| SEM | 0.003 | 0.002 | 0.003 | 0.003 | 0.003 | 0.002 | 0.003 | 0.003 |
| A/D0 (%) | — | −0.9 | +5.2 | +1.3 | — | −9.0* | +2.8 | −3.9 |
| p-value | — | 0.3788 | 0.9732 | 0.6491 | — | 0.0069 | 0.8191 | 0.1952 |

In comparison with placebo, the formula containing the Aureobasidium pullulans extract as an emulsion showed a significant improvement in skin microrelief after 14 days of treatment by reducing the Sa parameter by 8.1% (p=0.0120; effect observed in 65% of volunteers).

### Example 8: In Vitro Effect of the Active Principle in Accordance with the Invention Alone and in Combination with C-Glycoside

### A. Materials and Methods

The extract of Aureobasidium pullulans is that prepared in Example 1 above.

The effects of the Aureobasidium pullulans extract and C-β-D-xylopyranoside-2-hydroxypropane (C-Xyloside, Pro-Xylane™) and a combination thereof were evaluated on normal human epidermal keratinocytes (NHEK) by analysing the expression of markers by in situ immunofluorescence labelling and image analysis:
expression of the differentiation markers: Transglutaminase K (TGK), filaggrin and involucrin,
expression of the tight junction proteins: claudin-1, zonula occludens-1 (ZO-1) and occludin,

The compounds were tested at a non-cytotoxic concentration.
Normal human keratinocytes: NHEK, Bioalternatives reference K341 used at third passage.
Culture conditions: 37° C., 5% CO₂
Culture medium: Keratinocyte SFM (serum free medium) supplemented with Epidermal Growth Factor Pituitary extract
Test medium: Keratinocyte SFM (serum free medium)

### Compounds Tested

TABLE 12

| | | Intermediate | Test con- |
|---|---|---|---|
| Test compound | Appearance/storage | solution | centrations |
| Aureobasidium | liquid | 1% in | 0.03% |
| pullulans | storage at room | culture | 0.1% and |
| extract* | temperature | medium | 0.3% |
| | Protected from light | | |
| | and moisture | | |
| C-B-D-xylopyranoside- | liquid | 1% in | 0.03% |
| 2-hydroxypropane (C- | | culture | |
| Xyloside, Pro- | storage at room | medium | 0.1% and |
| xylaneTM)* | temperature | | 0.3% |

*the Aureobasidium pullulans extract of Example 1 was tested in the form of a mixture comprising 2.15% by weight of dry matter of Aureobasidium pullulans, 15% by weight of propylene glycol, qs water.
**C-β-D-xylopyranoside-2-hydroxypropane was tested in the form of a mixture comprising 35% by weight of active material of C-β-D-xylopyranoside-2-hydroxypropane, 25% by weight of propylene glycol, qs water.

Positive control for expression of the markers: TGK, filaggrin, involucrin, claudin-1, ZO-1 and occludin: CaCl₂) Sigma, ref. C₇₉₀₂, stored in 150 mM ultrapure water solution, concentration tested=1.5 mM
Positive control for expression of the marker: Collagen IV: TGF-β R&D Systems, ref. 240-B, stored in 20 μg/ml solution of 4 mM HCl/0.1% BSA, concentration tested=1.6 to 10 ng/ml.

### Cultures and Treatments

TGK, claudin-1, ZO-1, collagen IV:

The keratinocytes were seeded in 96-well plates and cultured in culture medium for 24 hours. The medium was then replaced with a test medium containing or not containing (control) the test compounds, the combination or the reference compound and the cells were incubated for 72 hours.

### Filaggrin:

The keratinocytes were seeded in a 96-well plate and grown in culture medium for 192 hours with renewal of the culture medium after 24 and 96 hours. The medium was then replaced with a test medium containing or not containing (control) the test compounds, the combination or the reference compound and the cells were incubated for 72 hours.

### Involucrin and Occludin:

The keratinocytes were seeded in 96-well plates and cultured in culture medium for 24 hours. The medium was then replaced with a test medium containing or not containing (control) the test compounds, the combination or the reference compound and the cells were incubated for 144 hours with a repeat treatment after 72 hours.

### In Situ Immunofluorescent Labelling and Image Analysis

### Fixed Cells

The cells were rinsed with PBS solution, fixed and permeabilized. The cells were then labelled using a specific primary antibody (Table 13). The primary antibody was then revealed using an appropriate fluorescent secondary antibody (Table 13) and the cell nuclei were stained in parallel using Hoechst 33258 solution (bisbenzimide, Sigma, ref. B1155).

TABLE 13

| Protein | Primary antibody | Secondary antibody |
|---|---|---|
| TGK | Anti-TGK (Anti-TGK) | GAR-Alexa 488 |
| | Proteintech, ref. 12912-3-AP | Invitrogen, ref. A11008 |
| Filaggrin | Anti-filaggrin | GAM-Alexa 488 |
| | Santa Cruz, ref. SC-66192 | Invitrogen, ref. A11001 |
| Involucrin | Anti-involucrin | GAM-Alexa 488 |
| | Sigma, ref. I9018 | Invitrogen, ref. A11001 |
| Claudin 1 | Anti-claudin 1 | GAM-Alexa 488 |
| | Santa Cruz, ref. sc-81796 | Invitrogen, ref. A11001 |
| ZO-1 | Anti-ZO-1 | GAM-Alexa 488 |
| | Invitrogen, ref. 33-9100 | Invitrogen, ref. A11001 |
| Occludin | Anti-occludin | GAM-Alexa 488 |
| | Invitrogen, ref. 33-1500 | Invitrogen, ref. A11001 |
| Collagen IV | Anti-collagen IV | GAM-Alexa 488 |
| | Chemicon, ref. MAB6308 | Invitrogen, ref. A11001 |

### Microscopic Observation and Image Analysis

Image acquisition (five photos/well) was performed using an INCell Analyzer™ 2200 (GE Healthcare, x20 objective).

The labelling was quantified by measuring the fluorescence intensity and normalizing the fluorescence intensity relative to the total number of cells (Digital data integration with Developer Toolbox 1.5 software, GE Healthcare).

### Data Processing

The raw data were analysed using the Microsoft Excel software.

Intergroup comparisons were performed using an unpaired Student's t-test. The statistical analysis may be interpreted if n≥5, however for n<5 the statistical values are indicative. The formulae used are:

The standard error of the mean is calculated according to the following formula:

$$sem = Sd / \text{?} \qquad [ formula 3 ]$$

$$\text{?} \text{indicates text missing or illegible when filed}$$

The standard error of the mean (sem) is a measure of the probable deviation of the sample relative to the true population mean. The sem is calculated as the sd (standard deviation) divided by the square root of the sample size.

The percentage viability is calculated according to the following formula:

$$Viability ( \% ) = ( {OD}_{sample} / {OD}_{control} ) \times 100 \qquad [ formula 4 ]$$

OD: optical density.

### B. Results

ns: 0.05, not significant
*: 0.01 to 0.05, significant
**: 0.001 to 0.01, very significant
***: <0.001, extremely significant

The results per marker are collated in Tables 14 to 21 below:

TABLE 14
Expression of occludin

| | | Integration | | Significance |
|---|---|---|---|---|
| Compound | Concentration | fluo | sd | vs control |
| Control | — | 100 | 11 | — |
| CaCl₂ | 1.5 mM | 296 | 27 | ** |
| Aureobasidium pullulans | 0.3% | 192 | 17 | ** |
| extract | | | | |
| C-β-D-xylopyranoside-2- | 0.3% | 78 | 7 | ns |
| hydroxypropane | | | | |
| (C-Xyloside, | | | | |
| Pro-Xylane ™) | | | | |
| Aureobasidium pullulans | 0.3% + 0.3% | 175 | 5 | ** |
| extract + | | | | |
| C-β-D-xylopyranoside-2- | | | | |
| hydroxypropane (C-Xyloside, | | | | |
| Pro-xylane ™) | | | | |

TABLE 15
Expression of ZO-1

| | | Integration | | Significance |
|---|---|---|---|---|
| Compound | Concentration | fluo | sd | vs control |
| Control | — | 100 | 3 | — |
| CaCl₂ | 1.5 mM | 119 | 4 | * |
| Aureobasidium pullulans | 0.03% | 96 | 1 | ns |
| extract | 0.1% | 93 | 14 | ns |
| | 0.3% | 100 | 9 | ns |
| C-β-D-xylopyranoside-2- | 0.03% | 111 | 3 | ns |
| hydroxypropane | 0.1% | 124 | 11 | ns |
| (C-Xyloside, | | | | |
| Pro-Xylane ™) | 0.3% | 127 | 12 | ns |
| Aureobasidium pullulans | 0.03% + 0.03% | 137 | 8 | * |
| extract + | 0.1% + 0.1% | 122 | 7 | * |
| C-β-D-xylopyranoside-2- | 0.3% + 0.3% | 130 | 9 | * |
| hydroxypropane (C-Xyloside, | | | | |
| Pro-xylane ™) | | | | |

TABLE 16
Expression of Claudin-1

| | | Integration | | Significance |
|---|---|---|---|---|
| Compound | Concentration | fluo | sd | vs control |
| Control | — | 100 | 13 | — |
| CaCl₂ | 1.5 mM | 200 | 16 | ** |
| Aureobasidium pullulans | 0.03% | 91 | 20 | ns |
| extract | 0.1% | 89 | 5 | ns |
| C-β-D-xylopyranoside-2- | 0.03% | 184 | 13 | * |
| hydroxypropane | 0.1% | 204 | 30 | * |
| (C-Xyloside, | | | | |
| Pro-Xylane ™) | | | | |
| Aureobasidium pullulans | 0.03% + 0.03% | 172 | 16 | * |
| extract + | 0.1% + 0.1% | 221 | 14 | ** |
| C-β-D-xylopyranoside-2- | | | | |
| hydroxypropane (C-Xyloside, | | | | |
| Pro-xylane ™) | | | | |

TABLE 17
Expression of Involucrin

| | | Integration | | Significance |
|---|---|---|---|---|
| Compound | Concentration | fluo | sd | vs control |
| Control | — | 100 | 8 | — |
| CaCl₂ | 1.5 mM | 126 | 4 | * |
| Aureobasidium pullulans | 0.1% | 132 | 5 | * |
| extract | 0.3% | 148 | 3 | ** |
| C-β-D-xylopyranoside-2- | 0.1% | 109 | 4 | ns |
| hydroxypropane | | | | |
| (C-Xyloside, | | | | |
| Pro-Xylane ™) | 0.3% | 110 | 6 | ns |
| Aureobasidium pullulans | 0.1% + 0.1% | 132 | 6 | * |
| extract + | 0.03% + 0.03% | 162 | 12 | * |
| C-β-D-xylopyranoside-2- | | | | |
| hydroxypropane (C-Xyloside, | | | | |
| Pro-xylane ™) | | | | |

TABLE 18
Expression of Filaggrin

| | | Integration | | Significance |
|---|---|---|---|---|
| Compound | Concentration | fluo | sd | vs control |
| Control | — | 100 | 11 | — |
| CaCl₂ | 1.5 mM | 159 | 14 | * |
| Aureobasidium pullulans | 0.1% | 157 | 13 | * |
| extract | 0.3% | 179 | 24 | * |
| C-β-D-xylopyranoside-2- | 0.1% | 114 | 11 | ns |
| hydroxypropane | 0.3% | 127 | 18 | ns |
| (C-Xyloside, | | | | |
| Pro-Xylane ™) | | | | |
| Aureobasidium pullulans | 0.1% + 0.1% | 157 | 17 | * |
| extract + | 0.03% + 0.03% | 198 | 7 | ** |
| C-β-D-xylopyranoside-2- | | | | |
| hydroxypropane (C-Xyloside, | | | | |
| Pro-xylane ™) | | | | |

TABLE 19
Expression of TGK

| | | Integration | | Significance |
|---|---|---|---|---|
| Compound | Concentration | fluo | sd | vs control |
| Control | — | 100 | 8 | — |
| CaCl₂ | 1.5 mM | 903 | 163 | ** |
| Aureobasidium pullulans | 0.03% | 84 | 8 | ns |
| extract | 0.1% | 93 | 6 | ns |
| | 0.3% | 113 | 40 | ns |
| C-β-D-xylopyranoside-2- | 0.03% | 124 | 22 | ns |
| hydroxypropane | 0.1% | 158 | 7 | ** |
| (C-Xyloside, | | | | |
| Pro-Xylane ™) | 0.3% | 235 | 45 | * |
| Aureobasidium pullulans | 0.03% + 0.03% | 246 | 28 | ** |
| extract + | 0.1% + 0.1% | 225 | 32 | * |
| C-β-D-xylopyranoside-2- | 0.3% + 0.3% | 388 | 42 | ** |
| hydroxypropane (C-Xyloside, | | | | |
| Pro-xylane ™) | | | | |

TABLE 20
Expression of Collagen IV

| | | Integration | | Significance |
|---|---|---|---|---|
| Compound | Concentration | fluo | sd | vs control |
| Control | — | 100 | 8 | — |
| TGF-β | 10 ng/ml | 176 | 9 | ** |
| Aureobasidium pullulans | 0.3% | 114 | 7 | ns |
| extract | | | | |
| C-β-D-xylopyranoside-2- | 0.3% | 120 | 12 | ns |
| hydroxypropane | | | | |
| (C-Xyloside, | | | | |
| Pro-Xylane ™) | | | | |
| Aureobasidium pullulans | 0.3% + 0.3% | 137 | 6 | * |
| extract + | | | | |
| C-β-D-xylopyranoside-2- | | | | |
| hydroxypropane (C-Xyloside, | | | | |
| Pro-xylane ™) | | | | |

Under the control conditions, the basal expression of the differentiation markers and the tight junction proteins TGK, filaggrin, involucrin, claudin-1, ZO-1 and occludin in NHEKs was either very low or limited to a small number of cells.

Treatment of NHEKs with the positive control CaCl₂) tested at 1.5 mM significantly stimulated the expression of TGK (903% of control), filaggrin (159% of control), involucrin (126% of control), claudin-1 (200% of control), ZO-1 (119% of control) and occludin (296% of control). These results were expected and validated the test.

As regards the dermoepidermal junction, the basal expression of collagen IV was very low. Treatment of NHEKs with TGF-β tested at 10 ng/ml significantly stimulated the collagen IV expression (176% of control). These results were expected and validated the test.

Under the experimental conditions of this test, the Aureobasidium pullulans extract induced a significant increase in the expression of filaggrin, involucrin and occludin.

The combination of the Aureobasidium pullulans extract and C-β-D-xylopyranoside-2-hydroxypropane (C-Xyloside, Pro-Xylane™) also stimulated the expression of the markers considered.

The main effects, which were slightly stronger than those of the Aureobasidium extract alone, were as follows:
a significant increase in collagen IV expression at the highest concentration of the combination, whereas alone, no efficacy was observed,
a synergistic increase in TGK, and
a significant increase in ZO-1 expression with a similar effect at the three concentrations tested, whereas alone, no efficacy was observed.

The result is that the Aureobasidium pullulans extract, optionally in combination with C-β-D-xylopyranoside-2-hydroxypropane, makes it possible to combat the signs of skin ageing and to improve the skin's barrier function.

### Example 9: Cosmetic Composition

The following lotion was prepared:

TABLE 21

| | % Concentrations |
|---|---|
| INCI name | (g/g) |
| Tetrasodium glutamate diacetate | 0.1 |
| Bis-PEG-18 methyl ether dimethyl silane | 1 |
| Rose floral water | 53 |
| Glycerol | 3 |
| Propanediol | 2 |
| Hydroxypropyl tetrahydropyrantriol (Proxylane) | 1.65 |
| Aureobasidium pullulans extract | 3 |
| obtained according to Example 1* | |
| Hydroxyethylpiperazineethanesulfonic acid | 5 |
| Xylityl glucoside (and) anhydroxylitol (and) | 0.55 |
| xylitol | |
| PPG-26-buteth-26 (and) hydrogenated castor | 3 |
| oil PEG-40 | |
| Fragrance | qs |
| Preserving agents | qs |
| Water | qs |

*the Aureobasidium pullulans extract of Example 1 is incorporated into the composition in the form of a mixture comprising 2.15% by weight of dry matter of Aureobasidium pullulans extract, 15% by weight of propylene glycol, qs water.

The lotion was applied to the skin of the face.

1-17. (canceled)

18. A method of treating keratin materials, the method comprising applying to the keratin materials a composition comprising:
(a) at least one extract of biomass of Aureobasidium pullulans yeast, wherein the (a) at least one extract comprises at least 25% sugars by weight, relative to the dry weight of the extracts (a).

19. The method of claim 18, wherein the keratin materials are selected from skin and the method of treating is for preventing and/or treating the signs of skin ageing and/or reinforcing and/or improving the skin's barrier function.

20. The method of claim 18, wherein the method of treating is for moisturizing the skin and/or for improving the quality of the skin surface.

21. The method of claim 18, wherein the Aureobasidium pullulans yeast is isolated from rose bushes.

22. The method of claim 18, wherein the Aureobasidium pullulans yeast is isolated from flowers and/or thorns and/or roots of Rosa sp.

23. The method of claim 18, wherein the at least one extract comprises at least 45% sugars by weight, relative to the dry weight of the extract.

24. The method of claim 18, wherein the sugars comprises at least 80% oligosaccharides by weight, relative to the dry weight of the sugars.

25. The method of claim 24, wherein the oligosaccharides are chosen from alpha-linked glucose oligosaccharides, and/or beta-linked glucose oligosaccharides, and wherein the oligosaccharides have a molar mass of less than 1800 Da.

26. The method of claim 18, wherein the composition is in liquid form or in solid form.

27. The method of claim 18, wherein the composition is prepared by a process comprising:
a. cultivating Aureobasidium pullulans in a culture medium to obtain a biomass,
b. dissolving at least 50 g/L of the biomass in water,
c. extracting sugars from the resulting solution,
d. heating the solution at a temperature between 80° C. and 90° C.,
e. separating soluble and insoluble phases and recovering the soluble phase,
f. purifying the soluble phase by molecular sorting with optional decolourization and deodorization, and
g. optionally concentrating the purified extract and sterilizing the extract by filtration.

28. The method of claim 18, further comprising: combining the extract with (b) at least one C-glycoside.

29. The method of claim 18, wherein the at least one C-glycoside has formula (I): 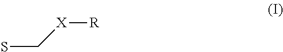
wherein:
R represents:
a saturated linear C₁ to C₂₀ or unsaturated C₂ to C₂₀, alkyl radical, or a saturated or unsaturated, branched or cyclic C₃ to C₂₀alkyl radical;
a saturated linear C₁ to C₂₀, or unsaturated C₂ to C₂₀, branched hydrofluoro- or perfluoroalkyl radical, or cyclic saturated or unsaturated C₃ to C₂₀, hydrofluoro- or perfluoroalkyl radical;
a phenyl or benzyl radical,
the hydrocarbon-based chain of R comprising said radicals possibly being, where appropriate, interrupted with 1, 2, 3 or more heteroatoms chosen from: an oxygen, a sulfur, a nitrogen, a silicon, or a halogen atom,
and possibly being optionally substituted with at least one radical chosen from: (i) —OR4, —SR4, —NR4R5, —COOR4, —CONHR4, —CN, a C1 to C6 hydrofluoro- or perfluoroalkyl radical; (ii) a C₃ to C₈ cycloalkyl radical; (iii) a C₅ to C₁₈ cycloalkyl, aryl or heterocyclic radical,
wherein R4 and R5 are independently chosen from a hydrogen atom, or a linear, saturated C1 to C30 or unsaturated C2 to C30, or branched or cyclic, saturated or unsaturated, C3 to C30, alkyl, perfluoroalkyl or hydrofluoroalkyl radical; or a C6 to C10 aryl radical,
X represents a radical chosen from —CO—, —CH(OH)—, or —CH(NH₂)—;
S represents a monosaccharide or a polysaccharide comprising up to 20 sugar units, in pyranose and/or furanose form and of L and/or D series, said mono- or polysaccharide possibly being substituted with a mandatorily free hydroxyl group, and optionally one or more optionally protected amine functions, and
S—CH₂—X represents a bond of C-anomeric nature, which may be α or β,
salts thereof, solvates thereof, hydrates thereof, or isomers thereof.

30. The method of claim 28, wherein C-glycoside is C-β-D-xylopyranoside-2-hydroxypropane.

31. The method of claim 28, wherein the weight ratio of the total amount of (b) C-glycosides to the total amount of (a) extracts of the biomass of Aureobasidium pullulans yeast is less than or equal to 50:1.

32. The method of claim 28, wherein a weight ratio of a total amount of C-glycosides to a total amount of extracts of the biomass of Aureobasidium pullulans yeast ranges from 8:1 to 49:1.

33. A cosmetic composition, comprising:
(a) at least one extract of Aureobasidium pullulans, and
(b) at least one C-glycoside,
wherein the weight ratio of the total amount of (b) C-glycosides to the total amount of (a) extracts of the biomass of Aureobasidium pullulans yeast is less than or equal to 50:1.

34. The composition of claim 33, wherein the total amount of (a) extracts ranges ranging from 0.001% to 1% by weight, relative to the total dry weight of the composition.

35. The composition of claim 33, in wherein the total amount of (b) C-glycoside ranges from 0.001% to 10% by weight, relative to the total dry weight of the composition.

36. A method for treating keratin materials, comprising applying to the keratin materials a cosmetic composition comprising:
(a) at least one extract of Aureobasidium pullulans, and
(b) at least one C-glycoside,
wherein the weight ratio of the total amount of (b) C-glycosides to the total amount of (a) extracts of the biomass of Aureobasidium pullulans yeast is less than or equal to 50:1.

37. The method of claim 36, wherein the method is a method for improving the radiance of the skin and/or for improving the uniformity of the complexion and/or for reducing the microrelief of the skin.
